SEQUENCE LISTING (18 sequences)

SEQ ID NO: 1 - protein, 528 aa, Artificial Sequence
  RQR8-2A-aGFP_GBP6-HNG-CD28TM-41BBZ exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGDVQLQESGGGSVQTGGSLRLSCAVSPYIGSRISLGWFRQAPGKVREGVALINSRDGSTYYADTVKGRFTISQGDANTVYLQMNSLKPEDTAIYYCAARWGQITDIQALAVASFPDWGQGTQVTVSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 651 aa, Artificial Sequence
  RQR8-2A-aCD19FMC63_LH-HNG-CD28TM-41BBZ exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 654 aa, Artificial Sequence
  RQR8-2A-aCD33glx_LH-HNG-CD28TM-41BBZ exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 963 aa, Artificial Sequence
  RQR8-2A-aGFP_GBP6-HNG-CD28TM-41BBZ-E2A-aGFP_kub4_VHH-L3-aCD33glx_ LH-H6 exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGDVQLQESGGGSVQTGGSLRLSCAVSPYIGSRISLGWFRQAPGKVREGVALINSRDGSTYYADTVKGRFTISQGDANTVYLQMNSLKPEDTAIYYCAARWGQITDIQALAVASFPDWGQGTQVTVSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQCTNYALLKLAGDVESNPGPMYRMQLLSCIALSLALVTNSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSDPSGGGGSGGGGSGGGGSDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSGGGGSHHHHHH

SEQ ID NO: 5 - protein, 957 aa, Artificial Sequence
  RQR8-2A-aGFP_GBP6-HNG-CD28TM-41BBZ-E2A-aGFP_kub4_VHH-L3-aCD19FMC6 3_HL-H6 exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGDVQLQESGGGSVQTGGSLRLSCAVSPYIGSRISLGWFRQAPGKVREGVALINSRDGSTYYADTVKGRFTISQGDANTVYLQMNSLKPEDTAIYYCAARWGQITDIQALAVASFPDWGQGTQVTVSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQCTNYALLKLAGDVESNPGPMYRMQLLSCIALSLALVTNSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSDPSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKASGGGGSHHHHHH

SEQ ID NO: 6 - protein, 20 aa, Artificial Sequence
  signal peptide
METDTLLLWVLLLWVPGSTG

SEQ ID NO: 7 - protein, 5 aa, Artificial Sequence
  linker peptide
SGGGG

SEQ ID NO: 8 - protein, 16 aa, Artificial Sequence
  linker peptide
SGGGGSGGGGSGGGGS

SEQ ID NO: 9 - protein, 16 aa, Artificial Sequence
  XTEN linker peptide
SGSETPGTSESATPES

SEQ ID NO: 10 - protein, 415 aa, Artificial Sequence
  aGFP_kub4_VHH-L3-aCD33glx_LH-H6 bi-specific protein
METDTLLLWVLLLWVPGSTGQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSDPSGGGGSGGGGSGGGGSDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSGGGGSHHHHHH

SEQ ID NO: 11 - protein, 409 aa, Artificial Sequence
  aGFP_kub4_VHH-L3-aCD19FMC63_HL-H6 bi-specific protein
METDTLLLWVLLLWVPGSTGQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSDPSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKASGGGGSHHHHHH

SEQ ID NO: 12 - protein, 285 aa, Artificial Sequence
  intracellular 3xMYC-XTEN_L-eGFP
EQKLISEEDLEQKLISEEDLEQKLISEEDLSGSETPGTSESATPESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - protein, 2316 aa, Artificial Sequence
  p210_BCR-ABL-3xMYC-XTEN_L-eGFP
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSKALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYSFCVSYVDSIQQMRNKFAFREAINKLENNLRELQICPATAGSGPAATQDFSKLLSSVKEISDIVQREQKLISEEDLEQKLISEEDLEQKLISEEDLSGSETPGTSESATPESMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 14 - protein, 651 aa, Artificial Sequence
  RQR8-2A-aEpCAM_MT110_LH-HNG-CD28TM-41BBZ exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 1196 aa, Artificial Sequence
  RQR8-2A-aP53_421_LH-HNG-CD28TM-41BBZ-E2A-aEpCAM_MT110_LH-L-aP53_R 175Hmut_7B9_HL-H6 exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGDVLMTQTPLTLSVTIGQPASISCKSSQSLLDSDGKTYLNWLLQRPGQSPKRLIYLVSKLDSGVPDRFTGSGSGTDFTLKINRVEAEDLGVYYCWQGTHSPLTFGAGTKLEIKRSGGGGSGGGGSGGGGSQVQLQQSGAELVRSGASVKLSCTASGFNIKDYYMHWVKQRPEQGLEWIGWIDPENGDTEYAPKFQGKATMTADTSSNTAYLQLSSLASEDTAVYYCNFYGDALDYWGQGTTVTVSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQCTNYALLKLAGDVESNPGPMYRMQLLSCIALSLALVTNSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSEVQLQQSGPELVKPGASVKISCKTSGYTFTEYTMHWMKQSHGKSLEWIGRINPYSGGTVYNQKFKGKATLTVDKSSSTAYMELRSLTSDDSAVYYCARWGGDYVTGGGTTLTVSSGGGGSGGGGSGGGGSDIVMTQSPSSLSVSGGEKVTMSCKSSQSLLNSGNQKSNLAWYQQKPGQPPKLLIYGASTRESGVPDRFAGSGSGTDFTLTISSVQAEDLAVYYCQNDHSYPLTFGGGTKLELKRSGGGGSHHHHHH

SEQ ID NO: 16 - protein, 1196 aa, Artificial Sequence
  RQR8-2A-aP53_421_LH-HNG-CD28TM-41BBZ-E2A-aEpCAM_MT110_LH-L-aCD19_ FMC63_HL-H6 exemplary sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGDVLMTQTPLTLSVTIGQPASISCKSSQSLLDSDGKTYLNWLLQRPGQSPKRLIYLVSKLDSGVPDRFTGSGSGTDFTLKINRVEAEDLGVYYCWQGTHSPLTFGAGTKLEIKRSGGGGSGGGGSGGGGSQVQLQQSGAELVRSGASVKLSCTASGFNIKDYYMHWVKQRPEQGLEWIGWIDPENGDTEYAPKFQGKATMTADTSSNTAYLQLSSLASEDTAVYYCNFYGDALDYWGQGTTVTVSSDPAEPKSPDKTHTCPPCPKDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQCTNYALLKLAGDVESNPGPMYRMQLLSCIALSLALVTNSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKASGGGGSHHHHHH

SEQ ID NO: 17 - protein, 15 aa, Artificial Sequence
  flexible linker
GGGGSGGGGSGGGGS

SEQ ID NO: 18 - protein, 5 aa, Artificial Sequence
  serine-glycine linker
GGGGS

(19) United States
(12) Patent Application Publication
(10) Pub. No.: US 2023/0265186 A1
(43) Pub. Date: Aug. 24, 2023

(54) CHIMERIC ANTIGEN RECEPTOR CELL

(71) Applicant: UCL Business Ltd, London (GB)

(72) Inventors: Martin Pulé, London (GB); Weng-Kit Cheung, London (GB)

(21) Appl. No.: 18/009,320

(22) PCT Filed: Jun. 9, 2021
(86) PCT No.: PCT/GB2021/051435

§ 371 Date: Dec. 8, 2022

(30) Foreign Application Priority Data
Jun. 9, 2020 (GB) 2008688.0

(51) Int. Cl. C07K 16/28; C07K 14/725; A61K 39/00; A61P 35/00; C12N 5/0783

(52) U.S. Cl. CPC C07K 16/2803; A61K 39/4611; A61K 39/4631; A61K 39/464412; A61P 35/00; C07K 14/7051; C12N 5/0638; A61K 2239/13; A61K 2239/29; C07K 2317/31; C07K 2317/622; C07K 2319/03; C07K 2319/33; C12N 2510/00

(57) ABSTRACT

The present invention relates to a cell which comprises a chimeric antigen receptor (CAR) comprising a binding domain which binds a first epitope of a tumour antigen; and a polynucleotide which encodes a bi-specific protein which comprises a first binding domain which binds a second epitope of said tumour antigen; and a second binding domain which binds a cell surface antigen. The present invention also provides CAR systems, nucleic acids, vectors, pharmaceutical compositions and pharmaceutical compositions for use in the treatment and/or prevention of disease.

### FIELD OF THE INVENTION

The present invention relates to chimeric antigen receptors (CARs) and bi-specific proteins and in particular to combinations of CARs and bi-specific proteins; to cells comprising said CAR and expressing said bi-specific protein; and in particular to approaches which enable CARs to target antigens which are not typically present on the cell surface of a cell. The present invention relates to pharmaceutical compositions comprising the cells and/or bi-specific protein in accordance with the present invention and their use in treating and/or preventing a disease.

### BACKGROUND TO THE INVENTION

The immune system plays a role in the development and growth of many different types of cancer. Several different immunotherapeutic strategies are being developed as potential therapies for these cancers. Immunotherapeutic strategies for modifying the immune system to recognise tumour cells and promote anti-tumour effector function include immune checkpoint blockade, adoptive cell therapy (ACT) with tumour-infiltrating lymphocytes or genetically modified T cells expressing transgenic T cell receptors (TCR) or CARs.

The introduction of CARs or transgenic TCRs to cells allows the generation of large numbers of cells specific to an antigen by ex vivo introduction of the nucleic acid encoding the CAR or TCR to peripheral blood cells e.g. peripheral blood T cells.

CARs are artificial receptors which graft the specificity of a monoclonal antibody onto a cell, such as a T-cell, that can be constructed by linking the variable regions of the antibody heavy (VH) and light chains (VL) to intracellular signalling chains alone or in combination with other signalling moieties. CARs recognise antigens which are presented on the tumour cell surface.

CAR T-cells engraft within patients, proliferate and home to sites of disease. CAR T-cells have shown activity against lymphoid malignancies including those that are refractory to standard therapies. Persistence of CAR T-cells protects against relapse. Together these features make CAR T-cell therapy an attractive modality with which to treat cancer.

The most successful results to date have been obtained in haematological malignancies with the use of CD19-targeted CAR T cell therapy and has received commercial approval from the Food and Drug Administration (FDA). CAR T-cells have also been shown to be effective in treating refractory cancers.

However, one major limitation of CAR T-cells currently is they cannot target intracellular antigens, for example, the intracellular antigens is inaccessible to the antigen binding domain of the CAR. This restricts the possibility for CARs to specifically target cancer since many intracellular antigens develop mutations, fusions or aberrant phosphorylation events which are highly selective for tumour cells over normal cells. Tumour cells are known to leak intracellular antigens (many of which comprise these mutations, fusion or aberrant phosphorylation events which are highly selective for tumour cells, into the tumour microenvironment).

One way of recognizing intracellular antigens is by using transgenic TCRs recognizing peptides in the context of MHC. For example, TCRs which recognise: peptides from proteins which are over-expressed; or peptides which comprise point mutations; fusion junctions and even phosphor-epitopes can be generated.

However, transgenic TCRs have several limitations. Firstly, transgenic TCRs have to be generated for different HLA types. Nearly all transgenic TCRs described and tested in clinical studies have been HLA-A2 restricted. Depending on the population, HLA-A2 prevalence is 40% or less. Secondly, presentation of peptide is completely dependent on a complex process of processing protein and presentation of peptides. This means that there is considerable opportunity for disruption of the necessary machinery. Targeting multiple epitopes does not solve this problem associated since all peptides are dependent on these pathways. A further limitation of using transgenic TCRs to target intracellular antigens is that specificities are difficult to generate, and pre-clinical data does not predict toxicity due to cross-reactivity.

An improved means of targeting intracellular antigens for immunotherapy is required.

### SUMMARY OF ASPECTS OF THE INVENTION

The present invention is based, at least in part, on the inventors’ realisation that dysregulated tumour cells leak small amounts of tumour antigen (such as intracellular tumour antigen) which can be targeted using immunotherapy approaches. For example, small amounts of tumour antigen may be released by the tumour cell due to dysregulated ER/Golgi and/or membrane trafficking. These tumour antigens are released into the microenvironment around the tumour and are present at low levels. Since these tumour antigens are not present on the cell surface (i.e. are not retained on the cell surface), it has not been possible to target them with standard CARs. Their low levels of expression may also present problems for efficaciously targeting these tumour antigens for therapy.

The present invention provides a therapeutic strategy for targeting these tumour antigens which are present at low levels in the tumour microenvironment. The strategy comprises the use of a combination of 1) an engineered cell which expresses a CAR; and 2) a bi-specific protein. Illustrative embodiments of the invention are shown in FIGS. 1-4.

The bi-specific protein comprises a first domain which binds a cell surface antigen in the tumour microenvironment (e.g. on the cell surface of a tumour cell); and a second domain which binds a tumour antigen. The engineered CAR cell expresses a CAR which binds the tumour antigen at an epitope distinct from that recognized by the bi-specific protein. The tumour antigen accumulates on the surface of the cell (e.g. tumour cell) as it is released from the tumour cell or from neighbouring cells via interactions with the bi-specific protein. The CAR then binds to the tumour antigen which is tethered by the bi-specific protein. Target cells which trap the antigen on their surface are lysed.

This therapeutic strategy can be utilised to target tumour antigens including but not limited to:
fusion proteins expressed by tumours (e.g. wherein the bi-specific protein recognises and binds to one fusion partner and the CAR recognizes and binds to the other fusion partner);
proteins comprising tumour specific mutations (e.g. wherein the bi-specific protein recognizes and binds to an epitope of the protein and the CAR recognizes and binds to the mutation, which may be a tumour specific mutation (or vice versa));
proteins which are aberrantly phosphorylated in tumours (e.g. wherein the bi-specific protein recognises and binds to an epitope of the protein and the CAR recognizes and binds to the epitope comprising the aberrant phosphorylation (or vice versa)).

The engineered cells and bi-specific proteins according to the present invention thereby may provide improved engineered cells for use as therapeutics.

In one aspect, the present invention provides a cell which comprises;
(i) a CAR comprising a binding domain which binds a first epitope of a tumour antigen; and
(ii) a polynucleotide which encodes a bi-specific protein which comprises:
   a first binding domain which binds a second epitope of said tumour antigen; and
   a second binding domain which binds a cell surface antigen.

The cell may be an engineered immune effector cell.

The bi-specific protein may be a secreted protein.

The binding domains which bind the first epitope of a tumour antigen and the second epitope of the tumour antigen may be non-competitive. Suitably, the binding domain of the CAR which binds a first epitope of the tumour antigen and the first binding domain of the bi-specific protein which binds a second epitope of the tumour antigen may be capable of binding to the same antigen at the same time.

The cell surface antigen may be a cell surface tissue antigen.

Suitably, the tumour antigen is not a cell surface tumour antigen, preferably said tumour antigen does not comprise a transmembrane domain or a lipid anchor such as a glycosylphosphatidylinositol (GPI)-anchor.

Suitably, the tumour antigen does not comprise a signal peptide.

The tumour antigen may be expressed at a higher level in the tumour compared with a corresponding, non-cancerous tissue, or the antigen may be tumour-specific.

The first and/or second epitope of the tumour antigen may comprise a tumour-specific mutation. The mutation may be selected from a substitution, insertion or deletion.

The tumour antigen may be a fusion protein. Suitably said fusion protein may comprise at least two domains, a first domain of the fusion protein may comprise a first epitope of the tumour antigen and a second domain of the fusion protein may comprise the second epitope of the tumour antigen. In other words, the binding domain of the CAR and the first binding domain of the bi-specific protein bind to different domains of the fusion protein or different fusion partners which comprise the fusion protein.

The first or second epitope of the tumour antigen may comprise a tumour-specific post-translational modification.

The tumour-specific post-translational modification may be phosphorylation, suitably one of the tumour antigen epitopes may be the phosphorylation site. For example, one of the tumour antigen epitopes may be the phosphorylation site (which is specific for the tumour) and a second tumour antigen epitope may be an epitope which is not at the phosphorylation site, or is not tumour specific.

The binding domain of the CAR may bind a first epitope of a tumour antigen which is specific to the tumour.

The first binding domain of the bi-specific protein may bind a second epitope of a tumour antigen which is specific to the tumour.

The cell may be an immune effector cell, such as an alpha-beta T cell, a NK cell, a gamma-delta T cell, a cytokine induced killer cell or a macrophage.

In another aspect, the present invention provides a nucleic acid construct which comprises:
(i) a first nucleic acid sequence which encodes a CAR comprising a binding domain which binds a first epitope of a tumour antigen; and
(ii) a second nucleic acid sequence which encodes a bi-specific protein which comprises:
   a first binding domain which binds a second epitope of said tumour antigen; and
   a second binding domain which binds an epitope of a cell surface tissue antigen.

The first and second nucleic acid sequences may be separated by a co-expression site.

In a further aspect, the present invention provides a kit of nucleic acid sequences comprising:
(i) a first nucleic acid sequence which encodes a CAR comprising a binding domain which binds a first epitope of a tumour antigen; and
(ii) a second nucleic acid sequence which encodes a bi-specific protein, which comprises:
   a first binding domain which binds a second epitope of said tumour antigen; and
   a second binding domain which binds an epitope of a cell surface tissue antigen.

In another aspect, the present invention provides a vector which comprises a nucleic acid construct according to the present invention.

In another aspect, the present invention provides a kit of vectors which comprises:
(i) a first vector which comprises a nucleic acid sequence which encodes a CAR comprising a binding domain which binds a first epitope of a tumour antigen; and
(ii) a second vector which comprises a nucleic acid sequence which encodes a bispecific protein, which comprises:
   a first binding domain which binds a second epitope of said tumour antigen; and
   a second binding domain which binds an epitope of cell surface tissue antigen.

In a further aspect, the present invention provides a pharmaceutical composition which comprises a plurality of cells according to the present invention, a nucleic acid construct according to the present invention, a first nucleic acid sequence and a second nucleic acid sequence according to the present invention; a vector according to the present invention or a first and a second vector according to the present invention.

In another aspect, the present invention provides a pharmaceutical composition according to the present invention for use in treating and/or preventing a disease.

The disease may be cancer.

In a further aspect, the present invention provides a method for making a cell according to the present invention, which comprises the step of introducing: a nucleic acid construct according to the present invention, a first nucleic acid sequence and a second nucleic acid sequence according to the present invention; a vector according to the present invention or a first and a second vector according to the present invention into the cell.

In another aspect, the present invention provides a CAR system comprising;
(i) a receptor component comprising a binding domain which binds a first epitope of a tumour antigen, a transmembrane domain and a signaling domain; and
ii) a bi-specific protein which comprises:
   a first binding domain which binds a second epitope of said tumour antigen; and
   a second binding domain which binds an epitope of a cell surface tissue antigen.

The system may comprise an engineered immune effector cell. For example, the cell may be an alpha-beta T cell, a NK cell, a gamma-delta T cell, a cytokine induced killer cell or a macrophage. Suitably, the engineered immune effector cell may express the receptor component.

The system may comprise an engineered immune effector cell. For example, the cell may be an alpha-beta T cell, a NK cell, a gamma-delta T cell, a cytokine induced killer cell or a macrophage. Suitably, the engineered immune effector cell may express the bi-specific protein. The receptor component and the bi-specific protein may be expressed by the same cell. For example, the bi-specific protein may be produced by the system or by a component within the system.

The bi-specific protein may be administered to the system. For example, the bi-specific protein may be produced outside of the system and subsequently introduced to the system.

In a further aspect, the present invention provides a bi-specific protein for use in treating cancer in combination with a CAR expressing cell, wherein:

the bi-specific protein comprises:
a first binding domain which binds a second epitope of said tumour antigen; and
a second binding domain which binds an epitope of a cell surface tissue antigen; and
the CAR comprises a binding domain which binds a first epitope of a tumour antigen.

In another aspect, the present invention provides a CAR expressing cell for use in treating cancer in combination with a bi-specific protein, wherein:

the CAR comprises a binding domain which binds a first epitope of a tumour antigen; and the bi-specific protein comprises:
a first binding domain which binds a second epitope of said tumour antigen; and
a second binding domain which binds an epitope of a cell surface tissue antigen.

### DESCRIPTION OF THE FIGURES

FIG. 1 - shows a schematic diagram of an aspect of the invention. In panel (a) tumour cells over-express certain antigens known as tumour antigens. The tumour cell releases small amounts of intracellular proteins for example through dysregulated ER/Golgi and membrane trafficking. Hence, a small amount of tumour antigens are present extracellularly in the tumour microenvironment. However, these tumour antigens are not attached to the tumour cell membrane and are present at low levels so cannot be targeted by traditional CAR T-cells which typically target cell surface antigens only. In panel (b) a general concept of the invention is shown schematically. A CAR T-cell according to the present invention has been modified to secrete a bi-specific protein. This bi-specific protein comprises a binding domain with specificity for a surface tissue antigen also expressed by the tumour in question and a binding domain with specificity for the tumour antigen which does not overlap with recognition of said antigen by the CAR. The secreted bi-specific protein results in tethering and concentration of the tumour antigen on the tumour cell surface allowing targeting by the CAR.

FIG. 2 - shows a schematic diagram of one embodiment of the present invention, wherein the tumour expresses a fusion protein which comprises at least two domains e.g. EML4-ALK, BCR/ABL or any other fusion protein such as those listed in Table 2. The bi-specific protein binds one fusion partner and tethers / amplifies it on the tumour cell surface. The CAR recognizes the other fusion partner. This increases specificity of the system as only fusion protein is amplified and recognized by the CAR T-cell.

FIG. 3 - shows a schematic diagram of one embodiment of the present invention, wherein the tumour expresses a protein which has a tumour-specific mutation. The bi-specific protein may bind a generic epitope of the protein in question and tether / amplify it on the tumour cell surface whilst the CAR specifically recognizes the mutation or vice versa. This increases the specificity of the system as only the mutated protein is both amplified and recognized by the CAR T-cell.

FIG. 4 - shows a schematic diagram of one embodiment of the present invention, wherein the tumour expresses a protein which is aberrantly phosphorylated. The bi-specific protein may bind an epitope of the protein distinct from the site of aberrant phosphorylation and the CAR may specifically recognize the phosphor-epitope or vice versa. This increases the specificity of the system as only the aberrantly phosphorylated protein is both amplified and recognized by the CAR T-cell.

FIG. 5 - shows EXOAMP in vitro targeting of CD33 positive and intracellular eGFP expressing cells. EXOAMP GFP targeting CAR lyses double positive CD33/eGFP expressing targets and spares single positive CD33 expressing targets. Effector T-cells expressing CAR; anti-CD33 CAR (aCD33glx-HNG), anti-GFP CAR (αGFP_GBP6-HNG), negative control CD19-CAR (αCD19FMC63) and non-transduced (NT); co-expressing either, a bispecific binder against GFP/CD33 (αGFP-HNG-αCD33 tandem scFv) and an irrelevant bispecific binder against GFP/CD19 (αGFP-HNG-αCD19) were co-cultured at a 1:1 effector to target ratio against CD33 expressing HL60 cells (see panel A), HL60 cells expressing intracellular eGFP protein (3xMYC-XTEN_L-eGFP) (see panel B) and HL60 cells expressing an intracellular chimeric GFP/BCL-ABL protein (p210.p210_BCR-ABL-3xMYC-XTEN_L-eGFP) (see panel C).

FIG. 6 - shows EXOAMP in vivo targeting of CD19 expressing NALM6 cells and intracellular expressing GFP NALM6 cells in a subcutaneous NOD scid gamma (NSG) mouse by a CD19-CAR and EXOAMP GFP-CAR expressing bispecific protein targeting GFP/CD19 and non-transduced (NT) T-cells. EXOAMP GFP CAR targets and lyses double positive CD19/eGFP expressing NALM6.3xMYC-XTEN_L-eGFP tumour, while sparing single positive NALM6 tumour in bilaterally flanked NSG mice. Panel A shows details of the timeline, cohorts, sampling and routes of injection in the in vivo NALM6 subcutaneous animal model. NSG mice were injected with 0.5×10^6 NALM6 and NALM6.3xMYC-XTEN_L-eGFP tumour on alternating hind flanks, and 5×10^6 CAR T-cells were injected intratumourally three days after. Tumour cells have been transduced to firefly luciferase/glycosylphosphatidylinositol anchored HA tag (Fluc_xRed.2A.HA-GPI) as a marker for tumour growth in vitro and in vivo via bioluminescent imaging. Panel B shows raw images from D3-21 and show tumour control of both tumours in the αCD19-CAR; and specific double positive NALM6.3xMYC-XTEN_L-eGFP tumour control in the EXOAMP GFP CAR. This data is further plotted with average radiance at the tumour site in panel C, demonstrating specific tumour control with the EXOAMP GFP-CAR only. The graphs on the left hand side indicate specific tumour average radiance of NALM6 and the graphs on the right hand side indicate specific tumour average radiance of NALM6.3xMYC-XTEN_L-eGFP respectively. The lines indicate average readings of each subject mouse/cohort.

FIG. 7 - shows EXOAMP in vivo targeting of CD19 expressing NALM6 cells and intracellular expressing GFP NALM6 cells in a subcutaneous NOD scid gamma (NSG) mouse by αCD19-CAR and EXOAMP GFP-CAR expressing either- bispecific protein targeting GFP/CD19 or an irrelevant protein targeting GFP/CD33 and non-transduced (NT) T-cells. EXOAMP GFP CAR +GFPxCD19 targets and lyses double positive CD19/eGFP expressing NALM6.3xMYC-XTEN_L-eGFP tumour, while sparing single positive NALM6 tumour in bilaterally flanked NSG mice. While the GFP-CAR +GFPxCD33 fails to control both tumours similarly to NT T-cells. Panel A shows details of timeline, cohorts, sampling and routes of injection in the in vivo NALM6 subcutaneous animal model. NSG mice were injected with 0.5×10^6 NALM6 and NALM6.3xMYC-XTEN_L-eGFP tumour on alternating sites - left hind flank and right shoulder, and 5×10^6 CAR T-cells were injected intratumourally three days after. Tumour cells have been transduced to express firefly luciferase/ glycosylphosphatidylinositol anchored HA tag (Fluc_xRed.2A.HA-GPI) as a marker for tumour growth in vitro and in vivo via bioluminescent imaging. Panel B shows raw images from D3-21 and show tumour control of both tumours in the αCD19-CAR; and specific double positive NALM6.3xMYC-XTEN_L-eGFP tumour control in the EXOAMP GFP CAR. The GFP-CAR _GFPxCD33 fails to control both tumours similarly to NT T-cells. This data is further plotted with average radiance at the tumour site in panel C, demonstrating specific tumour control with the EXOAMP GFP-CAR +GFPxCD19 only. The graphs on the left hand side indicate specific tumour average radiance of NALM6 and the graphs on the right hand side indicate specific tumour average radiance of NALM6.3xMYC-XTEN_L-eGFP. The lines indicate average readings of each subject mouse/cohort. The first mouse of cohort 2 (αCD19_FMC63- HNG) had to be culled due to weight loss before D21.

FIG. 8 - shows extracellular p53 protein as measured by a p53-speciific ELISA on in vitro cellular supernatant from colon cancer cell lines, and control cells; activated PBMCs and non-activated PBMCs. 1×10⁵ cells were plated on a 96F well plate in 200 ul media, and incubated for 48 hours before cellular supernatant was quantified for p53 protein. p53 protein can be detected in ⅚ of the colon cancer cell lines tested. CL-40 and SW1463 cell lines express mutant p53 harbouring a R248Q mutation. HT-29 express R273H mutant p53. LS123 express R175H mutant p53. RKO and LS174T express wild type p53. Readings are 7 biological replicates for HT-29, LS123 and LS174T cells, 6 biological replicates for CL-40, SW1463, activated and non-activated PBMCs and 1 replicate for RKO cells.

FIG. 9 - shows IL-2 cytokine release from T cells when incubated with target colorectal cancer cells HT29 (which express cell surface EpCAM and extracellular p53 mutant R273H) or LS123 (which express cell surface EpCAM and extracellular p53 mutant R175H) wherein the T cells from left to right of the bar chart:
are non-transduced (NT) cells;
express an anti-EpCAM CAR (anti-EpCAM_MT110);
express an anti-CD19 CAR (anti-CD19_FM63 CAR);
express a pantropic p53 CAR and secrete a bispecific binder against EpCAM and mutant p53 R175H (aP53_421 +BSB_MT110/7B9);
express a pantropic p53 CAR and secrete a bispecific binder against EpCAM and CD19 (aP53_421 +BSB_MT110/FMC63).

FIG. 10 - shows IFN_{Y} cytokine release from T cells when incubated with target colorectal cancer cells HT29 (which express cell surface EpCAM and extracellular p53 mutant R273H) or LS123 (which express cell surface EpCAM and extracellular p53 mutant R175H) wherein the T cells from left to right of the bar chart:
are non-transduced (NT) cells;
express an anti-EpCAM CAR (anti-EpCAM_MT110);
express an anti-CD19 CAR (anti-CD19_FM63 CAR);
express a pantropic p53 CAR and secrete a bispecific binder against EpCAM and mutant p53 R175H (aP53_421 +BSB_MT110/7B9);
express a pantropic p53 CAR and secrete a bispecific binder against EpCAM and CD19 (aP53_421 +BSB_MT110/FMC63).

FIG. 11 - shows in vitro real time cytotoxicity of pantropic p53 CAR (aP53_421) expressing T-cells secreting a bispecific binder against EpCAM and mutant p53 R175H (+BSB_MT110/7B9) against LS123 cells which express cell surface EpCAM, and harbour mutant p53 R175H. Control effectors include non-transduced cells (NT) and positive control CAR anti-EpCAM (aEpCAM_MT110). A) Real-time analysis of viable LS123 target cells over 120 hours in a co-culture of E:T 4:1 ratio (CAR: targets). Dashed line 0.5 normalized viable targets, normalised to t=0.. B) Hours to 50% killing of LS123 target cells, as compared to viable targets normalized to t=0. n/a non-applicable.

### DETAILED DESCRIPTION

### Chimeric Antigen Receptor (CAR)

A classical chimeric antigen receptor (CAR) is typically a chimeric type I trans-membrane protein which connects an extracellular antigen-recognizing domain (binder) to an intracellular signalling domain(s) (endodomain). The binder is typically a single-chain variable fragment (scFv) derived from a monoclonal antibody (mAb), but it can be based on other formats which comprise an antibody-like antigen binding site. A spacer domain is usually used to isolate the binder from the membrane and to allow it to position itself in a suitable orientation. A common spacer domain used is the Fc of IgG1. More compact spacers can suffice such as the stalk from CD8α or even just the IgG1 hinge alone, depending on the antigen. A trans-membrane domain anchors the protein in the cell membrane and connects the spacer to the endodomain.

Early CAR designs had endodomains derived from the intracellular parts of either the y chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal - namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be introduced into cells e.g. immune effector cells such as T cells using, for example, retroviral vectors. Lentiviral vectors may be employed. In this way, a large number of antigen-specific cells can be generated for adoptive cell transfer. When a CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

Suitably, the CAR according to the present invention may comprise the general format antigen binding domain-spacer-transmembrane domain-signalling domain. Suitably, the CAR according to the present invention may have the general format antigen binding domain-spacer-transmembrane domain-signalling domain.

Suitably, the CAR according to the present invention may comprise the general format: antigen binding domain-CD3. Suitably, the CAR according to the present invention may have the general format: antigen binding domain-CD3.

### Binding Domain

The binding domain (or antigen-binding domain) is the portion of the CAR or bi-specific protein which recognizes and binds antigen.

Numerous binding domains are known in the art, including those based on the antigen binding site of an antibody, an antibody fragment, antibody mimetics, and T-cell receptors.

Examples of antibody fragments capable of binding to a selected target, include Fv, ScFv, F(ab′) and F(ab′)2.

For example, the binding domain may comprise: a single-chain variable fragment (scFv) such as an scFv derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The binding domain may be a polypeptide having an antigen binding site which comprises at least one complementarity determining region (CDR). The binding domain may comprise 3 CDRs and have an antigen binding site which is equivalent to that of a domain antibody (dAb). The binding domain may comprise 6 CDRs and have an antigen binding site which is equivalent to that of a classical antibody molecule. The remainder of the polypeptide may be any sequence which provides a suitable scaffold for the binding site and displays it in an appropriate manner for it to bind the antigen. The binding domain may be part of an immunoglobulin molecule such as a Fab, F(ab)′2, Fv, single chain Fv (ScFv) fragment, Nanobody or single chain variable domain (which may be a VH or VL chain, having 3 CDRs). The binding domain may be non-human, chimeric, humanised or fully human.

The binding domain may comprise a binding domain which is not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The binding domain may “specifically bind” to the antigen as defined herein. As used herein, “specifically bind” means that the binding domain binds to the antigen but does not bind to other proteins, or binds at a lower affinity to other proteins.

The binding affinity between two molecules, e.g. an antigen binding domain and an antigen, may be quantified, for example, by determination of the dissociation constant (KD). The KD can be determined by measurement of the kinetics of complex formation and dissociation between the binding domain and antigen, e.g. by a surface plasmon resonance (SPR) method (e.g. BiacoreTM). The rate constants corresponding to the association and the dissociation of a complex are referred to as the association rate constants ka (or kon) and dissociation rate constant kd. (or koff), respectively. KD is related to ka and kd through the equation KD = kd / ka. Binding affinities associated with different molecular interactions, e.g. comparison of the binding affinity of different binding domains and an antigen, may be compared by comparison of the KD values for the individual binding domain and antigen.

The binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The binding domain may be based on a natural ligand of the antigen.

The binding domain may comprise an affinity peptide from a combinatorial library or a de novo designed affinity protein/peptide.

Antibody fragments capable of binding to a selected target, include Fv, ScFv, F(ab′) and F(ab′)2. In addition, alternatives to classical antibodies may also be used, for example “avibodies”, “avimers”, “anticalins”, “nanobodies” and “DARPins”.

In one aspect, the binding domains which bind the first epitope of a tumour antigen and the second epitope to the tumour antigen may be non-competitive. Suitably, the binding domain of the CAR which binds a first epitope of the tumour antigen and the first binding domain of the bispecific protein which binds a second epitope of the tumour antigen may be capable of binding to the same antigen at the same time.

Various binding domains which bind to suitable antigens are known in the art. For example,

Tables 1-4 below lists exemplary commercial antibodies which comprise binding domains which may be used in the present invention.

Table 1. Commercially available antibodies comprising binding domains which bind to phospho antigens e.g. which bind to an epitope comprising the phospho residue or which bind to an epitope which is distinct from the phospho epitope.

TABLE 1

| | | | | |
|---|---|---|---|---|
| Antigen | Epitope; phospho residue | Antibody clone | Cat # | Supplier |
| BRAF | T401 | EPR2208Y | ab68215 | Abcam plc |
| | S729 | EPR2207 | ab124794 | Abcam plc |
| | S729 | FQS3318 | DCABH-1608 | Creative Diagnostics |
| | T401 | FQS3319Z | DCABH-9451 | Creative Diagnostics |
| | T401 | JJ08-72 | MA5-32430 | Thermo Fisher Scientific (Life Technologies) |
| | S445 | Ser445 | 2696S | Cell Signaling Technology, Inc. |
| | A peptide corresponding to the residues near the region-terminus of human BRAF was used as the immunogen | RM308 | R20328 | NSJ Bioreagents |
| Aurora A (AURKA) | Raised against amino acids 1-130 mapping the N-terminus of human origin | C1 | sc-398814 | Santa Cruz Biotechnology, Inc. |
| | Raised against amino acids 1-130 mapping the N-terminus of human origin | A-11 | sc-514374 | Santa Cruz Biotechnology, Inc. |
| | Immunogen was a partial recombinant protein comprising amino acids 2-110 of AURKA | 6G9 | DMABT-H13111 | Creative Diagnostics |
| | T288 | C39D8 | 3079S | Santa Cruz Biotechnology, Inc. |
| | T288 | F.131.2 | PIMA5149 04 | Thermo Fisher Scientific (Life Technologies) |
| | T288 | 95.Thr 288 | sc-293126 | Santa Cruz Biotechnology, Inc. |

Table 2. Commercially available antibodies comprising binding domains which bind to a tumour fusion protein e.g. which bind to an epitope on one of the fusion partners.

TABLE 2

| | | | | |
|---|---|---|---|---|
| Tumour fusion | Antigen | Antibody clone | Cat # | Supplier |
| EML4-ALK | EML4 | W18139A | 670801 | BioLegend, Inc. |
| | | D7Y8F | 12156 | Cell Signaling Technology, Inc. |
| | | #2428 | 2428S | Cell Signaling Technology, Inc. |
| | | 2F2 | SAB1401649-100UG | Sigma-Aldrich Ltd |
| | | 3C10 | WH0027436M1-100UG | Sigma-Aldrich Ltd |
| | ALK (CD246) | SP144 | ab183332 | Abcam plc |
| | | SP8 | ab16670 | Abcam plc |
| | | 5A4 | ab17127 | Abcam plc |
| | | OTI1A4 | UM800118 | OriGene Technologies, Inc. |
| | | ALK1 | MABC1171-100UG | Sigma-Aldrich Ltd |
| | | OTI1H7 | TA800712 | OriGene Technologies, Inc. |
| | | OTI1D9 | CF801141 | OriGene Technologies, Inc. |
| CCDC6-RET | CCDC6 | 5D11 | WH0008030M3-100UG | Sigma-Aldrich Ltd |
| | | Q-23 | sc-100309 | Santa Cruz Biotechnology, Inc. |
| | | 6E22 | CABT-B9907 | Creative Diagnostics |
| | RET | 4C3 | ABIN6950046 | antibodies-online Inc. |
| | | 8D10C9 | ABIN230698 | antibodies-online Inc. |
| | | RET-2663 | ABIN6940506 | antibodies-online Inc. |
| | | 132507 | ABIN308572 | antibodies-online Inc. |
| | | 3F8 | ABIN384274 | antibodies-online Inc. |
| NCOA4-RET | NCOA4 | 2H8 | ABI N563528 | antibodies-online Inc. |
| | | C-4 | sc-373739 | Santa Cruz Biotechnology, Inc. |
| KIF5B-RET | KIF5B | EPR10276(B) | ab167429 | Abcam plc |

Table 3. Commercially available antibodies comprising binding domains which bind to antigens with point mutations e.g. which bind to an epitope comprising the point mutation or which bind to an epitope which is distinct from the point mutation (referred to as a wild-type epitope).

TABLE 3

| | | | | |
|---|---|---|---|---|
| Antigen | Epitope | Antibody clone | Cat# | Supplier |
| TP53 | - N-terminal epitope mapping between residues 11-25 | DO-1 | sc-126 | Santa Cruz Biotechnology, Inc. |
| | Raised against synthetic peptide corresponding to amino acid residues 371-380 | Pab421 | ab245685 | Abcam |
| | Amino acids 212-217 | Pab240 | sc-99 | Santa Cruz Biotechnology, Inc. |
| | Amino acids 1-45 | DO-7 | sc-47698 | Santa Cruz Biotechnology, Inc. |
| | N-terminus between amino acids 32-79 | Pab1801 | sc-98 | Santa Cruz Biotechnology, Inc. |
| | Raised against the N-terminus of p53 | Bp53-12 | sc-263 | Santa Cruz Biotechnology, Inc. |
| | Raised against amino acids 10-16 | DO-2 | sc-53394 | Santa Cruz Biotechnology, Inc. |
| | R175H mutant | XH117 | DMAB-DCC108 | Creative Diagnostics |
| | R282W mutant | XH119 | DMAB-DCC109 | Creative Diagnostics |
| KRAS | Raised against a synthetic peptide corresponding to N-terminal amino acids 1-100 | EP1125 Y | ab52939 | Abcam plc |
| | Raised against amino acids 1-188 | 3B10-2F2 | WH0003845M1-100UG | Sigma-Aldrich Ltd |
| | Raised against amino acids 16-125 | 4F3 | SAB1404011-100UG | Sigma-Aldrich Ltd |
| | Raised against amino acids 54-189 | F234 | sc-30 | Santa Cruz Biotechnology, Inc. |
| | Raised against amino acids 1-186 | 2F8 | NBP2-59413-50ul | Novus Biologicals |
| | A146T mutant | XH105 | DMAB-DCC097 | Creative Diagnostics |
| | G12D mutant | XH106 | DMAB-DCC098 | Creative Diagnostics |
| | G12S mutant | XH107 | DMAB-DCC099 | Creative Diagnostics |
| | G13A mutant | XH108 | DMAB-DCC100 | Creative Diagnostics |
| | G13D mutant | XH109 | DMAB-DCC101 | Creative Diagnostics |
| | Q61H mutant | XH110 | DMAB-DCC102 | Creative Diagnostics |
| | Q61L mutant | XH111 | DMAB-DCC103 | Creative Diagnostics |
| | Q61R mutant | XH112 | DMAB-DCC104 | Creative Diagnostics |
| | G12D mutant | D8H7 | 14429S | Cell Signaling Technology, Inc. |
| | G12V mutant | D2H12 | 14412S | Cell Signaling Technology, Inc. |
| BRAF | Raised against amino acids 50-150 | EP152Y | ab33899 | Abcam plc |
| | Raised against amino acids 346-445 | 3C6 | WH0000673M1-200UL | Sigma-Aldrich Ltd |
| | Raised against amino acids 346-445 | 3C2 | SAB1403617-100UG | Sigma-Aldrich Ltd |
| | D594G mutant | XH015 | DMAB-DCC011 | Creative Diagnostics |
| | G469E mutant | XH016 | DMAB-DCC012 | Creative Diagnostics |
| | V600D mutant | XH018 | DMAB-DCC014 | Creative Diagnostics |
| | N581S mutant | XH017 | DMAB-DCC013 | Creative Diagnostics |
| | V600E mutant | XH019 | DMAB-DCC015 | Creative Diagnostics |
| | V600K mutant | XH020 | DMAB-DCC016 | Creative Diagnostics |
| | V600R mutant | XH021 | DMAB-DCC017 | Creative Diagnostics |
| | V600E mutant | SN9 | CABT-BL8457 | Creative Diagnostics |
| | V600E mutant | RM8 | ABIN6560108 | antibodies-online Inc. |
| | V600E mutant | K21-F | ABIN2207472 | antibodies-online Inc. |
| | Raised against amino acids 550-650 (comprising V600E mutation) | VE1 | ab228461 | Abcam plc |
| PIK3CA | Raised against recombinant fragment. | 4F3 | SAB5300225-100UG | Sigma-Aldrich Ltd |
| | Raised against synthetic peptide corresponding to C-terminal amino acids 1000 onwards | EP383Y | ab40776 | Abcam plc |
| | Raised against amino acids 959-1068 | 3G3 | WH0005290M1-100UG | Sigma-Aldrich Ltd |
| | E542Q | XH095 | DMAB-DCC089 | Creative Diagnostics |
| | E542K | XH094 | DMAB-DCC088 | Creative Diagnostics |

Table 4. Commercially available antibodies comprising binding domains which bind to antigens which are over expressed in tumours.

TABLE 4

| | | | | |
|---|---|---|---|---|
| Antigen | Epitope | Antibody clone | Cat | Supplier |
| PRAME | 100aa-end | EPR20330 | ab219650 | Abcam plc |
| | 126-205aa | D-12 | sc-166480 | Santa Cruz Biotechnology, Inc. |
| | 126-205aa | H-10 | sc-137188 | Santa Cruz Biotechnology, Inc. |
| Survivin (BIRC5) | Raised against 1-100aa | 5B10 | WH0000332M 1-100UG | Sigma-Aldrich Ltd |
| | Raised against 1-142aa | D-8 | sc-17779 | Santa Cruz Biotechnology, Inc. |
| | 2-29aa at the N-terminus | C-6 | sc-374616 | Santa Cruz Biotechnology, Inc. |
| | 57-67aa | 3C184 | ABIN2369687 | antibodies-online Inc. |
| WT1 | A domain 180 aa in length mapping near the N-terminus | F-6 | sc-7385 | Santa Cruz Biotechnology, Inc. |
| | 314-479aa | 5G11A5 | ABIN5684073 | antibodies-online Inc. |
| | outside exon 2 | WT1-857 | ABIN5590995 | antibodies-online Inc. |
| | outside exon 2 | WT1-1434R | ABIN5590998 | antibodies-online Inc. |
| | Raised against recombinant protein corresponding to amino acids 1-181aa | SPM361 | ABIN5590992 | antibodies-online Inc. |
| | 123-164aa | H-1 | sc-393498 | Santa Cruz Biotechnology, Inc. |
| | Raised against recombinant protein corresponding to amino acids 1-181aa | 6F-H2 | ABIN5590990 | antibodies-online Inc. |
| | 195-317aa | 960525 | ABIN5666563 | antibodies-online Inc. |
| Telomerase reverse transcriptase (TERT) | 1087-1113aa near the C-terminus | A-6 | sc-393013 | Santa Cruz Biotechnology, Inc. |
| | Raised against 900-1130aa | C-12 | sc-377511 | Santa Cruz Biotechnology, Inc. |
| | Raised against 1029-1132aa | 3H2C12 | ABIN5542472 | antibodies-online Inc. |
| | Raised against 1029-1132aa | 3H2G9 | ABIN5954350 | antibodies-online Inc. |
| | Raised against 1100-1200aa | 1C1 | ABIN6950409 | antibodies-online Inc. |

Suitably, binding domains for use in any aspect of the present invention may be based on binding domains from the commercially available antibodies listed in Table 1-4. Suitably, binding domains for use in any aspect of the present invention may comprise the binding domains of any of the commercially available antibodies listed in Tables 1-4.

In one aspect, a binding domain for use in any aspect of the present invention may be based (or comprise) an antibody against mutant p53. Suitably, the antibody may be a mutation-specific antibody i.e. the antibody binds to mutant p53. Exemplary antibodies which may be used in the present invention are described in WO2018074978 and Hwang et al., 2018, Cell Reports 22, 299-312 which are both incorporated herein by reference. In one aspect, a binding domain for use in any aspect of the present invention may be based on (or comprise) an antibody against mutant p53 comprising R175H. The binding domain for use in any aspect of the present invention may be based on (or comprise) an antibody which is specific for a mutant p53 comprising R175H.

In one aspect, a binding domain for use in any aspect of the invention may be based on (or comprise) antibody clone 7B9.

In one aspect, a binding domain for use in any aspect of the present invention may be based on (or comprise) an antibody against mutant p53 comprising R248Q. The binding domain for use in any aspect of the present invention may be based on (or comprise) an antibody which is specific for a mutant p53 comprising R248Q. Exemplary antibodies are described in WO2018074978 and Hwang et al.,supa.

In one aspect, a binding domain for use in any aspect of the present invention may be based on (or comprise) an antibody against mutant p53 comprising R273H. The binding domain for use in any aspect of the present invention may be based on (or comprise) an antibody which is specific for a mutant p53 comprising R273H. Exemplary antibodies are described in WO2018074978 and Hwang et al.,supa.

### Spacer Domain

The CAR may comprise a spacer sequence to connect the binding domain with the transmembrane domain and spatially separate the binding domain from the endodomain.

The bi-specific protein may comprise a spacer sequence between the two binding domains. Suitably, the spacer sequence may spatially separate the two binding domains of the bi-specific protein.

A flexible spacer allows the binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

### Transmembrane Domain

The transmembrane domain is the sequence of the CAR that spans the membrane.

Suitably, the CAR may be a single-span protein.

Suitably, the CAR may be a multi-span protein.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention.

The presence and span of a transmembrane domain of a protein can be predicted by those skilled in the art using bioinformatics tools such as the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (for example as described in US 7052906 B1 which is incorporated herein by reference).

The transmembrane domain may be derived from CD28, which gives good receptor stability. The transmembrane domain may be derived from a component of the TCR receptor complex. The transmembrane domain may be derived from a TCR alpha chain. Suitably, the transmembrane domain may comprise a TCR alpha chain.

The transmembrane domain may be derived from a TCR beta chain. Suitably, the transmembrane domain may comprise a TCR beta chain.

The transmembrane domain may be derived from a CD3 chain. Suitably, the transmembrane domain may comprise a CD3 chain.

Suitably, the transmembrane domain may be derived from a CD3-epsilon chain. Suitably, the transmembrane domain may comprise a CD3-epsilon chain.

Suitably, the transmembrane domain may be derived from a CD3-gamma chain. Suitably, the transmembrane domain may comprise a CD3-gamma chain.

Suitably, the transmembrane domain may be derived from a CD3-delta chain. Suitably, the transmembrane domain may comprise a CD3-delta chain.

Suitably, the transmembrane domain may be derived from a CD3-zeta chain. Suitably, the transmembrane domain may comprise a CD3-zeta chain.

### Activating Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative / survival signal, or all three can be used together.

Where a CAR comprises an activating endodomain, it may comprise the CD3-Zeta endodomain alone, the CD3-Zeta endodomain with that of either CD28 or OX40 or the CD28 endodomain and OX40 and CD3-Zeta endodomain.

Any endodomain which contains an ITAM motif can act as an activation endodomain.

Suitably, the CAR according to the present invention may be a split receptor, such that the antigen recognition domain is a separate protein from the signalling domain.

The activating endodomain may be a TCR intracellular domain.

Suitably, the activating endodomain may comprise a stimulatory domain from an intracellular signalling domain from a component of the TCR receptor complex.

The activating endodomain may be derived from a component of the TCR receptor complex. The activating endodomain may be derived from a CD3 chain. Suitably, the activating endodomain may comprise a CD3 chain.

Suitably, the activating endodomain may be derived from a CD3-epsilon chain. Suitably, the activating endodomain may comprise a CD3-epsilon chain.

Suitably, the transmembrane domain may be derived from a CD3-gamma chain. Suitably, the transmembrane domain may comprise a CD3-gamma chain.

Suitably, the activating endodomain may be derived from a CD3-delta chain. Suitably, the activating endodomain may comprise a CD3-delta chain.

Suitably, the activating endodomain may be derived from a CD3-zeta chain. Suitably, the transmembrane domain may comprise a CD3-zeta chain.

### Bi-Specific Protein

A “bi-specific protein” as used herein refers to a protein which comprises a first binding domain which binds an epitope of a tumour antigen; and a second binding domain which binds a cell surface antigen.

In one aspect the bi-specific protein is located in the extracellular space. The bi-specific protein may be an extracellular protein. For example, the bi-specific protein may be located in the extracellular space of the tumour microenvironment.

In one aspect the bi-specific protein is a secreted protein.

The term “secreted protein” as used herein refers to any protein which is found in the extracellular space. This includes, for example, proteins which are secreted from cells through the classical secretory pathway and proteins which are secreted from cells through non-classical (or leaderless, non-conventional or unconventional) secretory pathways.

In one aspect, the bi-specific secreted protein is secreted from a cell through the classical secretory pathway. The bi-specific protein for use in the present invention may comprise a signal peptide so that when it is expressed in a cell, such as a T-cell, the nascent protein is directed to the endoplasmic reticulum and subsequently to the cell surface, where it is released from the cell. Classical protein secretion may be predicted using Signal P and TargetP methods Nielsen,H., et al., (1997) Protein Eng., 10, 1-6; Emanuelsson,O., Nielsen,H., Brunak,S. and von Heijne,G. (2000) J. Mol. Biol., 300, 1005-1016), which are incorporated herein by reference.

Signal peptides are typically 16 to 30 amino acids in length and are usually found at the N-terminus of the newly synthesized protein. The core of the signal peptide may contain a stretch of hydrophobic amino acids (about 5 to about 16 amino acids in length) that has a tendency to form a single alpha-helix. The signal peptide may begin, at the N terminus, with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

An example of a signal peptide which may be used is:

METDTLLLWVLLLWVPGSTG (SEQ ID NO: 6)

In another aspect, the bi-specific secreted protein is secreted from a cell through the non-classical secretory pathway. Non-classical protein secretion may be predicted using the SecretomeP 2.0 server, J. Dyrløv Bendtsen, et al., Protein Eng. Des. Sel., 17(4):349-356, 2004 (which is incorporated herein by reference). Examples of proteins which may be secreted without an N-terminal signal peptide include FGF-1, FGF-2, IL-1 and galectins. In one aspect the bi-specific secreted protein for use in the present invention does not comprise an N-terminal signal peptide.

In one aspect, the bi-specific secreted protein is expressed by a cell, such as an immune effector cell. The bi-specific secreted protein may be expressed by a cell which expresses a receptor component comprising a binding domain which binds a first epitope of a tumour antigen, a transmembrane domain and a signalling domain. The bi-specific secreted protein may be expressed by a cell which expresses a CAR comprising a binding domain which binds a first epitope of a tumour antigen as described herein.

In one aspect, the bi-specific protein is introduced or administered to the extracellular space. The bi-specific protein may be introduced or administered to the extracellular space in the tumour microenvironment. For example, the bi-specific protein may be introduced to the tumour microenvironment directly by injection or by systemic administration to the subject.

The bi-specific protein may be a soluble protein. Suitably, the bi-specific protein may be a soluble protein in the extracellular space of the tumour microenvironment.

As used herein “soluble” means that the bi-specific protein is capable of moving around the tumour microenvironment.

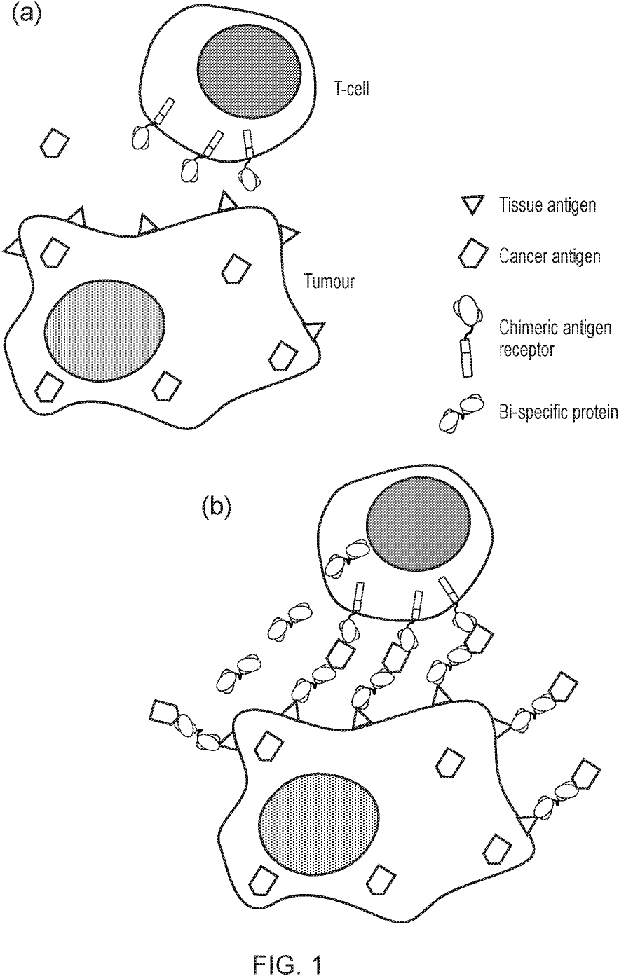

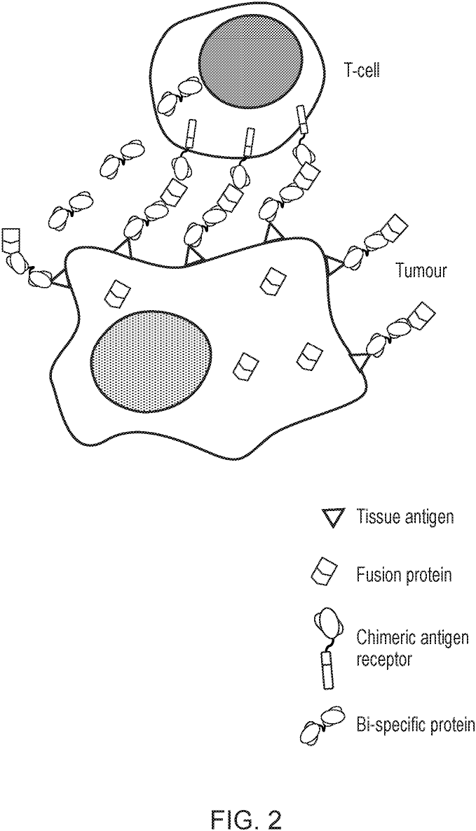

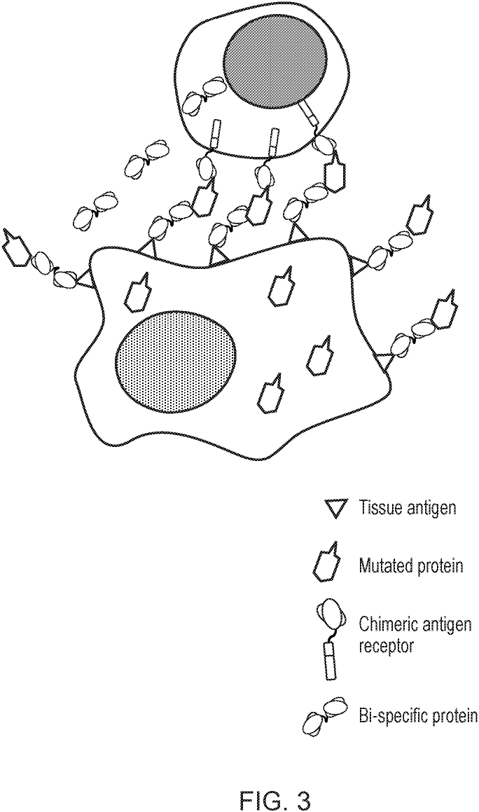

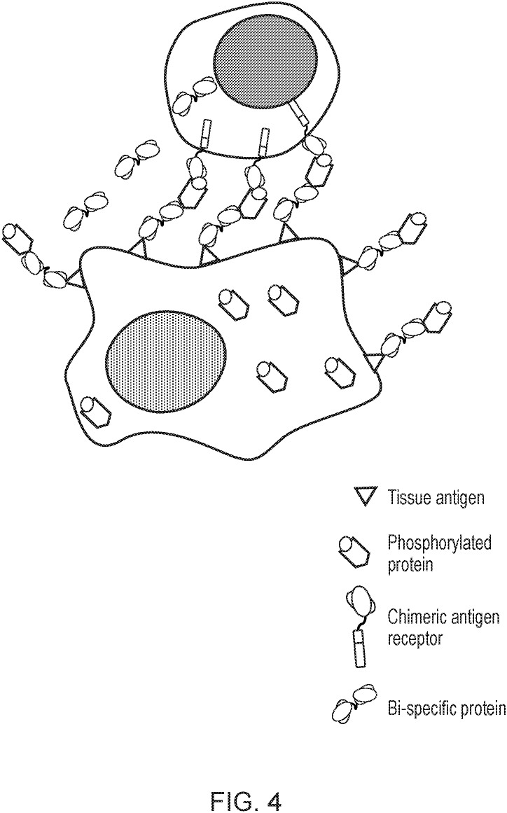

Suitably, prior to binding antigen, the bi-specific protein may not be directly or indirectly tethered to a cell surface. Thus both ends of the bi-specific protein are available to bind antigens. Once the bi-specific protein binds one of its target antigens, it becomes indirectly tethered to the cell surface. See for example, FIG. 1b). Once the bi-specific protein binds both of its target antigens, it becomes indirectly tethered to the cell surface of both the cell comprising a CAR and the tumour cell. See for example, FIG. 1b).

The binding domains of the bi-specific protein may be connected to one another by any suitable means. For example, the binding domains may be directly fused to one another. Alternatively, the bi-specific protein may comprise a spacer domain or linker between the binding domains. The linker provides flexibility for example, to enable the bi-specific protein to bind to the tumour antigen and to the cell surface antigen.

Suitably, the spacer domain or linker may spatially separate the two binding domains of the bi-specific protein. The linker may be a peptide linker.

Suitable linker peptides are known in the art. For example, a range of suitable linker peptides are described by Chen et al., (Adv Drug Deliv Rev. 2013 October 15; 65(10): 1357-1369, which is incorporated herein by reference - see Table 3 in particular).

A suitable linker is an (_{SGGGG})n (SEQ ID NO: 7), which comprises one or more copies of SEQ ID NO: 7. For example, a suitable linker peptide is shown as SEQ ID NO: 8.

SGGGGSGGGGSGGGGS (SEQ ID NO: 8).

Another exemplary linker is XTEN linker:

SGSETPGTSESATPES (SEQ ID NO: 9)

Exemplary sequences of bispecific protein and/or domains for use in bispecific binders according to the present invention include:

aGFP_kub4_VHH-L3-aCD33glx_LH-H6 (SEQ ID NO: 10):

METDTLLLWVLLLWVPGSTGQVQLVESGGALVQPGGSLRLSCAASGFPVN RYSMRWYRQAPGKEREWVAGMSSAGDRSSYEDSVKGRFTISRDDARNTVY LQMNSLKPEDTAVYYCNVNVGFEYWGQGTQVTVSSDPSGGGGSGGGGSGG GGSDIQMTQSPSSLSASVGDRVTITCRASEDIYFNLVWYQQKPGKAPKLL IYDTNRLADGVPSRFSGSGSGTQYTLTISSLQPEDFATYYCQHYKNYPLT FGQGTKLEIKRSGGGGSGGGGSGGGGSGGGGSRSEVQLVESGGGLVQPGG SLRLSCAASGFTLSNYGMHWIRQAPGKGLEWVSSISLNGGSTYYRDSVKG RFTISRDNAKSTLYLQMNSLRAEDTAVYYCAAQDAYTGGYFDYWGQGTLV TVS SGGGGSHHHHHH

The domains in the sequence above are in order:
Signal peptide
aGFP kub4 VHH
L3 serine-glycine linker
aCD33qlx LH scFv
Hexahistidine tag

aGFP_kub4_VHH-L3-aCD19FMC63_HL-H6 (SEQ ID NO: 11):

METDTLLLWVLLLWVPGSTGQVQLVESGGALVQPGGSLRLSCAASGFPVN RYSMRWYRQAPGKEREWVAGMSSAGDRSSYEDSVKGRFTISRDDARNTVY LQMNSLKPEDTAVYYCNVNVGFEYWGQGTQVTVSSDPSGGGGSGGGGSGG GGSEVKLQESGPGLVAPSQSLSVTCTVSGVSLPDYGVSWIRQPPRKGLEW LGVIWGSETTYYNSALKSRLTIIKDNSKSQVFLKMNSLQTDDTAIYYCAK HYYYGGSYAMDYWGQGTSVTVSSGGGGSGGGGSGGGGSDIQMTQTTSSLS ASLGDRVTISCRASQDISKYLNWYQQKPDGTVKLLIYHTSRLHSGVPSRF SGSGSGTDYSLTISNLEQEDIATYFCQQGNTLPYTFGGGTKLEITKASGG
GGSHHHHHH

The domains in the sequence above are in order:
Signal peptide
aGFP kub4 VHH
L3 serine-glycine linker
aCD19FMC63_HL
Hexahistidine tag

An illustrative bi-specific protein may comprise a sequence as shown in SEQ ID NO: 10 or 11 or a variant thereof with at least 80% (such as at least 85%, at least 90%, at least 95%, at least 97%, at least 99%) identity to any of SEQ ID NO: 10 or 11 provided that the variant protein is capable of acting as a bi-specific protein. Suitably, a bi-specific protein may comprise one or more domains as shown above from SEQ ID NO: 10 or 11 or a variant thereof with at least 80% (such as at least 85%, at least 90%, at least 95%, at least 97%, at least 99%) identity to domains within SEQ ID NO: 10 or 11 provided that the variant protein is capable of acting as a bi-specific protein.

### Tumour Antigen

### Expression

As used herein, “tumour antigen” refers to an antigen produced by tumour cells.

In one aspect, the tumour antigen is expressed at a higher level by the tumour compared with a corresponding, non-cancerous tissue.

Suitably, the tumour antigen may be expressed at a level which is at least 20% higher, at least 30% higher, at least 40% higher, at least 50% higher, at least 60% higher, at least 70% higher, at least 80% higher, at least 90% higher than in a corresponding non-cancerous tissue. Suitably, the tumour antigen may be tumour-specific.

As used herein “tumour specific” means that the antigen is not expressed or is expressed at a lower amount in a non-cancerous cell of the same lineage.

Suitably, the tumour antigen may be expressed at a level which is at least 25%, at least 30%, at least 40%, at least 50%, at least 60%, at least 70%, at least 80%, at least 90%, at least 95% lower in a non-cancerous cell of the same lineage when compared to the tumour.

Suitably, the tumour antigen may not be expressed in a corresponding, non-cancerous tissue. The level of expression may be calculated as a percentage of cells which are positive for the cell surface tissue antigen, for example by flow cytometry. A comparison may be made between a population of cells taken from the tumour and a population of cells from a corresponding non-cancerous tissue or a population of non-cancerous cells of the same lineage.

In one aspect, the tumour antigen comprises at least one epitope which is tumour specific. Said tumour specific epitope may be recognised by an antigen binding domain of the CAR or the bispecific protein. For example the epitope which is tumour specific may comprise a mutation, a fusion domain or an aberrant post-translation modification such as phosphorylation.

In one embodiment, the tumour antigen is overexpressed when compared with a corresponding non-cancerous tissue of the same lineage.

Suitably, the tumour antigen which is “overexpressed” may be expressed at a level which is at least 20%, at least 30%, at least 40%, at least 50%, at least 60%, at least 70%, at least 80%, at least 90%, at least 95% higher in the tumour when compared with a non-cancerous cell of the same lineage.

Suitably, the tumour antigen may be selected from: PRAME; Survivin; WT1; Telomerase; MDM2; Kalliikrein 4 and ERG.

Suitably, the tumour antigen may be any of PRAME; Survivin; WT1 or Telomerase.

Cancers which are associated with overexpression of these tumour antigens are shown in Table 5 below.

TABLE 5

Tumour antigens and cancers
Overexpressed/tumour antigens

| | |
|---|---|
| Target gene | Cancer |
| PRAME | Melanoma. Sarcoma. Leukaemia. Central nervous system (CNS). Kidney. |
| | Ovarian. Endometrial. Lung. Head & Neck. Liver. Breast. Kidney. Prostate. Soft tissue. |
| Survivin | Sarcoma. CNS. Breast. Cervical. Colorectal. Endometrial. Gastric. Head & neck. Liver. Kidney. Leukaemia. Lung. Mesothelioma. Melanoma. Ovarian. Pancreatic. Prostate. |
| WT1 | Lung. Breast. Gastric. Head & Neck. Colorectal. Sarcoma. Thyroid. Neuroblastoma. Leukaemia. Ovarian. Endometrial. Pancreas. |
| Telomerase | Bladder. Kidney. Liver. Melanoma. Sarcoma. Thyroid. CNS. Breast. Head & neck. Leukaemia. Pancreas. |
| MDM2 | Sarcoma. Breast. Lung. Colorectal. Soft tissue. |
| Kallikrein 4 | Prostate. Ovarian. |
| ERG | Prostate. Sarcoma. Soft tissue. Acute myeloid leukaemia (AML). T-cell acute lymphoblastic leukaemia) T-ALL. |

### Localisation

Most cancer targets are proteins which are not expressed on the surface of tumour cells. Proteins which are found on the cell surface (such as transmembrane proteins or GPI-linked proteins) may be targeted using standard CARs but proteins which are found inside the cell cannot be targeted by standard CARs. The present invention enables targeting of proteins which are typically found inside the cell, thereby increasing the number of tumour antigens which may be amendable to CAR therapy.

In cancer, tumour cells release small amounts of intracellular proteins for example, through dysregulated ER/Golgi and membrane trafficking, thereby releasing small amounts of tumour antigens to the tumour microenvironment. The present invention provides a method for using these tumour antigens as targets for CAR therapy by capturing them on the cell surface using the combination of a CAR and a bi-specific protein according to the present invention.

In one aspect, the tumour antigen is not a cell surface tumour antigen.

As used herein, “cell surface tumour antigen” refers to a tumour antigen which is expressed on the surface of a cell. In other words, a cell surface tumour antigen is exposed to the extracellular space.

Suitably, the tumour antigen does not: comprise a transmembrane domain; or partially span the phospholipid bilayer, or have a lipid anchor such as a glycosylphosphatidylinositol (GPI)-anchor. In one aspect, the tumour antigen is not a secreted protein. Suitably, the tumour antigen does not comprise a signal peptide.

In one aspect, the tumour antigen is not an extracellular protein.

In one aspect, the tumour antigen is an intracellular protein. As used herein “intracellular protein” means that the protein is expressed inside the cell.

In one aspect, the tumour antigen is a TCR antigen.

By “TCR antigen” it is meant any antigen which can be targeted by a TCR. For example, the tumour antigen may be selected from: WT1; MAGE; A3; P53; NY-ESO-1; CEA; MART1; GP100; Proteinase3; Tyrosinase; Survivin; hTERT or EphA2.

In one aspect, the tumour antigen is a protein which is predominantly an intracellular protein.

By “predominantly an intracellular protein”, it is meant that at least 50%, at least 60%, at least 70%, at least 80%, at least 90% of the tumour antigen is intracellular, or in other words is expressed within the cell.

In one aspect, the tumour antigen is a protein which is predominantly an intracellular protein in a corresponding non-cancerous cell from the same lineage. In one aspect, the tumour antigen is a protein which is predominantly an intracellular protein in the tumour cell.

Various methods exist for determining the subcellular localisation of proteins are well known in the art and include for example: electron microscopy; confocal microscopy; immunofluorescence; and flow cytometry with fluorescently tagged antibodies.

### Mutation

Genome-wide analysis has shown that solid tumours typically contain 20-100 protein-encoding genes that are mutated (Stratton MR, Campbell PJ, Futreal PA Nature. 2009 Apr 9; 458(7239):719-24). A fraction of these are considered to be “drivers” responsible for initiation or progression of tumours whilst the remainder are “passengers” providing no selective growth advantage. However both types of mutation provide opportunities for targeting tumour cells.

In one aspect, the tumour antigen comprises a mutation.

Examples of mutations which may be targeted using the present invention can be found in the Cosmic database: https://cancer.sanger.ac.uk/cosmic/ (J. Tate et al., COSMIC: the Catalogue of Somatic Mutations in Cancer: Nucleic Acids Research, Volume 47, issue D1, 8 Jan. 2019, Pages D941-D947, which is incorporated herein by reference).

The mutation may be a tumour-specific mutation.

Suitably, the first or second epitope of the tumour antigen may comprise a tumour-specific mutation.

Suitably, the mutation may be any type of mutation, for example the mutation may be selected from a substitution, insertion or deletion.

In one embodiment, the mutation may be a point mutation.

For example, the point mutation may be found in any of the following genes: TP53; KRAS; BRAF; PTEN; BRCA1; BRCA2; ATM; CDKN2A or PIK3CA.

Suitably, the point mutation may be found in any of the following genes: TP53; KRAS; BRAF or PTEN. Suitably, the point mutation may be found in TP53, such as R175H.

Exemplary mutations in tumour antigens and associated cancers are shown in Table 6 below.

TABLE 6

Examples of tumour antigens comprising mutations and associated cancers

| | | |
|---|---|---|
| Target gene | Cancer | Mutations |
| TP53 | Skin. Lung. Breast. Colorectal. Prostate. Gastric. Live. Cervical. Head & Neck. Bladder. Pancreatic. Ovarian. Oesophageal. CNS. Leukaemia. Lymphoma. | R175H, G245S, R248Q, R248W, R249S, R273H, R273C, R282W, Y220C |
| BRCA1 | Breast. Ovarian. Colorectal. Cervical. Uterus. Pancreas. Prostate. | S1577P |
| BRCA2 | Breast. Ovarian. Colorectal. Cervical. Uterus. Pancreas. Prostate. Gallbladder. Gastric. Bone. | G2044V, S2303F |
| ATM | Breast. Leukamia. Lymphoma. Lung. Brain. Pancreas. | R250, R337C, R337H, R3008H, T935R, P2453H, R2832C |
| KRAS | Pancreatic. Colorectal. Lung. Prostate. Skin. Thyroid. Liver. Ovarian. Endometrial. Renal. CNS. Testicular. Leukaemia/Lymphoma. Bladder. Head & Neck. Breast. | G12C, G12R, G12S, G12A, G12D, G12V, G13D, G13C, G13V, Q61H, Q61R, A146T |
| BRAF | Melanoma. Colorectal. Ovarian. Lung. Breast. Liver. Thyroid. CNS. Prostate. Renal. Pancreatic. Endometrial. | V600E, G469A, G469V, K601E, D594N, D594G, N581S |
| CDKN2 A | Skin. Pancreatic. Head & Neck. Lung. Ovarian. Gastric. Prostate. | G101W, V126D, H66R, H83Y, P114L |
| PIK3CA | Pancreatic. Colorectal. Lung. Prostate. Skin. Thyroid. Liver. Ovarian. Endometrium. Renal. Brain. Testicular. Leukemia. Bladder. Head & Neck. Breast. Gastric. | H1047R, E545K, E542K, N345K, R88Q, C420R |
| PTEN | Skin. Lung. Breast. Colorectal. Prostate. Gastric. Live. Cervical. Head & Neck. Bladder. Pancreatic. Ovarian. Oesophageal. CNS. Leukaemia. Lymphoma. | F341V, R130Q, R173C, R130G, R173H |

### Fusion Protein

Fusion proteins occur when a complex mutation, such as a chromosomal translocation, tandem duplication, or retrotransposition creates a novel coding sequence containing parts of the coding sequences from two different genes. Fusion proteins are commonly found in cancerous tumour cells. Such fusion proteins may function as oncoproteins. For example, the bcr-abl fusion protein is a well-known oncogenic fusion protein, and is considered to be the primary oncogenic driver of chronic myelogenous leukemia (CML). Examples of fusion proteins which may be targeted using the present invention can be found in the Cosmic database: https://cancer.sanger.ac.uk/cosmic/fusion (J. Tate et al., supra, which is incorporated herein by reference).

In one aspect, the tumour antigen is a fusion protein.

Suitably, the tumour antigen may be a fusion protein and may be selected from: EML4-ALK; CCD6-RET; NCOA4-RET; KIF5B-RET; KIF5B-ALK; TMPRSS2-ERG; EWSR1-FLI1; SYT-SSX; PAX3-FOXO1; TMPRSS2-ETV1 or PAX7-FOXO1.

Suitably, the tumour antigen may be a fusion protein and may be selected from: EML4-ALK; CCD6-RET; NCOA4-RET; KIF5B-RET or KIF5B-ALK.

Suitably the fusion protein may comprise at least two domains. A first domain may comprise the first epitope of a tumour antigen and a second domain may comprise the second epitope of the tumour antigen. For example each binding partner may recognise a different fusion partner of the fusion protein. Exemplary fusion proteins and associated cancers are shown in Table 7 below.

TABLE 7

Examples of fusion proteins as tumour antigens and associated cancers

| | |
|---|---|
| Target gene | Cancer |
| EML4-ALK | Lung cancer. Breast. Colorectal. |
| CCDC6-RET | Lung. Colorectal. Thyroid. ALL. |
| TMPRSS2-ERG | Prostate. Sarcoma. Soft tissue. AML. T-ALL. |
| EWSR1-FLI1 | Ewings Sarcoma |
| NCOA4-RET | Lung. Breast. Colorectal |
| KIF5B-RET | Lung |
| KIF5B-ALK | Lung |
| SYT-SSX | Synovial Sarcoma |
| PAX3-FOXO1 | Sarcoma. Soft tissue |
| TM PRSS2-ETV1 | Prostate. |
| PAX7-FOXO1 | Sarcoma. Soft tissue |

### Post-Translational Modification

It is known that many cellular processes are regulated by the reversible reaction of protein phosphorylation on serine, threonine and tyrosine residues. Disruption of this signal transduction cascade has been implicated in many diseases, including cancer. The importance of phosphorylation on a molecular level has been implicated specifically within signalling pathways involved in the pathogenesis of cancer. New phosphoproteomic technologies have identified new biomarkers comprising post-translational modifications, which present new targets for therapeutic approaches.

For example, MHC-class 1 associated phosphopeptides are the targets of memory like immunity in leukaemia. Examples of phosphopeptide which may be targeted using the present invention may be found in Cobbald et al., Sci Transl Med. 2013 Sep 18; 5(203): 203ra125, which is incorporated herein by reference.

In one aspect, the tumour antigen comprises a post-translational modification.

The post-translational modification may be tumour-specific.

Suitably, the first or second epitope of the tumour antigen may comprise a post-translational modification.

Suitably, the post-translational modification may be any type of post-translational modification, for example it may be phosphorylation.

Suitably, the tumour antigen may comprise a post-translational modification and may be selected from: KRAS; BRAFT; AuroraA; ERG; PIK3R1; ALK; mTOR; RET; RB1; ABL1; ABL2 or ROS1.

Suitably, the tumour antigen may comprise a post-translational modification and may be selected from: KRAS; BRAFT; AuroraA; ERG or PIK3R1.

Suitably, the first or second epitope of the tumour antigen may comprise a post-translational site, such as a phosphorylation site. Exemplary phosphoantigens, phosphor residues and associated cancers are shown in Table 8 below.

TABLE 8

Phosphoantigens, phospho residues and associated cancers

| | | |
|---|---|---|
| Target gene | Cancer | Phospho residue |
| KRAS | Pancreatic. Colorectal. Lung. Prostate. Skin. Thyroid. Liver. Ovarian. Endometrium. Renal. Brain. Testicular. Leukemia. Bladder. Head & Neck. Breast. Gastric. | S181 |
| BRAF | Melanoma. Colorectal. Ovarian. Lung. Breast. Liver. Thyroid. CNS. Prostate. Renal. Pancreatic. Endometrial. | T599, S602 |
| PIK3R 1 | Pancreatic. Colorectal. Lung. Prostate. Skin. Thyroid. Liver. Ovarian. Endometrium. Renal. Brain. Testicular. Leukemia. Bladder. Head & Neck. Breast. Gastric. | Y688 |
| Aurora A | Breast. Ovarian. Pancreatic. Bladder. Esophageal/Nasopharygeal. Colorectal. Gastric. Prostate. Liver. Renal. Multiple Myeloma. | T288, T287 |
| ALK | Lung cancer. Breast. Colorectal. | Y1278, Y1282, Y1283, Y1358, Y1507, Y1604 |
| ERG | Prostate. Sarcoma. Soft tissue. AML. T-ALL. | S96, S215 |
| mTOR | Lung. Gastric. Breast. Colorectal. Renal. Bladder. Prostate. Head & Neck. | S2448, S2481, S1261, T2446 |
| RET | Thyroid. Lung. Colorectal. | Y900, Y905, Y981, Y1062, Y687, Y928 |
| RB1 | Skin. Bladder. Lung. Breast. Bone. Head & Neck. Leukaemia. Soft tissue. | S807, S811 |
| ABL1 | Chronic myeloid leukaemia (CML). | Y177, Y412, Y245 |
| ABL2 | Breast. Ovarian. Lung. | Y439, Y272 |
| ROS1 | Lung. Endometrial. Gastric. Colorectal. Glioblastoma. Renal. Melanoma | Y591, Y651 |

### EXEMPLARY SEQUENCES

Exemplary sequences and/or domains for use according to the present invention include:

RQR8-2A-aGFP_GBP6-HNG-CD28TM-41BBZ (SEQ ID NO: 1):

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLLTCGDVEENPGPMETDTLLLWVLLLWVPGSTGDVQ LQESGGGSVQTGGSLRLSCAVSPYIGSRISLGWFRQAPGKVREGVALINS RDGSTYYADTVKGRFTISQGDANTVYLQMNSLKPEDTAIYYCAARWGQIT DIQALAVASFPDWGQGTQVTVSSDPAEPKSPDKTHTCP PCPKDPKFWVLV VVGGVLACYSLLVTVAFIIFWVRSKRGRKKLLYIFKQPFMRPVQTTQEED GCSCRFPEEEEGGCELRVKFSRSADAPAYQQGQNQLYNELNLGRREEYDV
LUKRRGRDFEMGGKPRRKNPQEGLYNELQKDKMAEAYSEIGMKGERRRGK
GHDGLYQGLSTATKDTYDALHMQALPPR

The domains in the sequence above are in order:
RQR8 marker gene
2A
Signal peptide
aGFP GBP6
Hinge linker (HNG)
CD28 transmembrane
41BB endodomain
CD3Z endodomain

RQR8-2A-aCD19FMC63_LH-HNG-CD28TM-41BBZ (SEQ ID NO:
2):

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSDDTCGDVEENPGPMETDTLLLWVLLLWVPGSTGDIQ MTQTTSSLSASLGDRVTISCRASQDISKYLNWYQQKPDGTVKLLIYHTSR LHSGVPSRFSGSGSGTDYSLTISNLEQEDIATYFCQQGNTLPYTFGGGTK LEITKAGGGGSGGGGSGGGGSGGGGSEVKLQESGPGLVAPSQSLSVTCTV SGVSLPDYGVSWIRQPPRKGLEWLGVIWGSETTYYNSALKSRLTIIKDNS KSQVFLKMNSLQTDDTAIYYCAKHYYYGGSYAMDYWGQGTSVTVSSDPAE PKSPDKTHTCP PCPKDPKFWVLVVVGGVLACYSLLVTVAFIIFWVRSKRG RKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNE
LQKDKMAEAYSEIGMKGERRRGKHDGLYQGLSTATKDTYDALHQALPPR

The domains in the sequence above are in order:
RQR8 marker gene
2A
Signal peptide
aCD19FMC63 LH
Hinge linker (HNG)
CD28 transmembrane
41BB endodomain
CD3Z endodomain

RQR8-2A-aCD33glx_LH-HNG-CD28TM-41BBZ (SEQ ID NO: 3
):

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLLTCGDVEENPGPMAVPTQVLGLLLLWLTDARCDIQ MTQSPSSLSASVGDRVTITCRASEDIYFNLVWYQQKPGKAPKLLIYDTNR LADGVPSRFSGSGSGTQYTLTISSLQPEDFATYYCQHYKNYPLTFGQGTK LEIKRSGGGGSGGGGSGGGGSGGGGSRSEVQLVESGGGLVQPGGSLRLSC AASGFTLSNYGMHWIRQAPGKGLEWVSSISLNGGSTYYRDSVKGRFTISR DNAKSTLYLQMNSLRAEDTAVYYCAAQDAYTGGYFDYWGQGTLVTVSSMD
PAEPKSPDKTHTCP PCPKDPKFWVLVVVGGVLACYSLLVTVAFIIFWVRS KRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGL
YNELQKDKMAEAYSEIQLYNELNLGRREEYDVLDKRRGRDPEMGGKPRRK
NPQEGLYNELQKDKMAEAYSEIGMKGERRRGKGHDGLYQGLSTATKDTYD
AKLHMQALPPR

The domains in the sequence above are in order:
RQR8 marker gene
2A
Signal peptide
aCD33glx LH
Hinge linker (HNG)
CD28 transmembrane
41BB endodomain
CD3Z endodomain

RQR8-2A-aGFP-GBP6_HNG-CD28TM-41BBZ-E2A-aGFP_kub4_V
HH-L3-aCD33glx_LH-H6(SEQ ID NO: 4):

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLL TCGDVEENPGFMETDTLLLWVLLLWVPGSTGDV QLQES GGGSVQTGGS LRLSCAVSPYIGSRISLGWFRQAPGKVREGVAL INSRDGSTYYADTVKGRFTISQGDANTVYLQMNSLKPEDTAIYYCAARWG QITDIQALAVASFPDWGQGTQVTVSSDPAEPKSPDKTHTCP PCPKDPKFW VLVVVGGVLACYSLLVTVAFIIFWVRSKRGRKKLLYIFKQPFMRPVQTTQ EEDGCSCRFPEEEEGGCELRVKFSRSADAYQQGQNQLYNELNLGRREEYD
VLDKRRGRDPEMGGKPRRKNPQEGLYNELQKDKMAEAYSEIGMKGERRRG
HDGLYQGLSTATKDTYDALHMQALPPRQCTNYALLKLAGDVESNPGPMYR
MQLLSCIALSLALVTNSQVQLVESGGALVQPGGSLRLSCAASGFPVNRYS MRWYRQAPGKEREWVAGMSSAGDRSSYEDSVKGRFTISRDDARNTVYLQM NSLKPEDTAVYYCNVNVGFEYWGQGTQVTVS SDPSGGGGSGGGGSGGGGS DIQMTQSPSSLSASVGDRVTITCRASEDIYFNLVWYQQKPGKAPKLLIYD TNRLADGVPSRFSGSGSGTQYTLTISSLQPEDFATYYCQHYKNYPLTFGQ GTKLEIKRSGGGGSGGGGSGGGGSGGGGSRSEVQLVESGGGLVQPGGSLR LSCAASGFTLSNYGMHWIRQAPGKGLEWVSSISLNGGSTYYRDSVKGRFT ISRDNAKSTLYLQMNSLRAEDTAVYYCAAQDAYTGGYFDYWGQGTLVTVS
SGGGGSHHHHHH

The domains in the sequence above are in order:
RQR8 marker gene
2A
Signal peptide
aGFP GBP6
Hinge linker (HNG)
CD28 transmembrane
41BB endodomain
CD3Z endodomain
E2A
Signal peptide
aGFP kub4
L3 serine-glycine linker
aCD33glx LH
Hexahistidine tag

RQR8-2A-aGFP_GBP6-HNG-CD28TM-41BBZ-E2A-aGFP_kub4_V
HH-L3-aCD19FMC63_HL-H6 (SEQ ID NO: 5):

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLL TCGDVEENPGFMETDTLLLWVLLLWVPGSTGDV QLQES GGGSVQTGGS LRLSCAVSPYIGSRISLGWFRQAPGKVREGVAL INSRDGSTYYADTVKGRFTISQGDANTVYLQMNSLKPEDTAIYYCAARWG QITDIQALAVASFPDWGQGTQVTVSSDPAEPKSPDKTHTCP PCPKDPKFW VLVVVGGVLACYSLLVTVAFIIFWVRSKRGRKKLLYIFKQPFMRPVQTTQ EEDGCSCRFPEEEEGGCELRVKFSRSADAPAYQQGQNQLYNELNLGRREE
YDVLDKRRGRDPEMGGKPRRKNPQEGLYNEELQKDKMAEAYSEIGMKGER
RRGKGHDGLYQGLSTATKDTYDALHMQALPPRQCTNYALLKLA GDVESN PGPMYRMQLLSCIALSLALVTNSQVQLVESGGALVQPGGSLRLSCAASGF PVNRYSMRWYRQAPGKEREWVAGMSSAGDRSSYEDSVKGRFTISRDDARN TVYLQMNSLKPEDTAVYYCNVNVGFEYWGQGTQVTVS SDPSGGGGSGGGG SGGGGSEVKLQESGPGLVAPSQSLSVTCTVSGVSLPDYGVSWIRQPPRKG LEWLGVIWGSETTYYNSALKSRLTIIKDNSKSQVFLKMNSLQTDDTAIYY CAKHYYYGGSYAMDYWGQGTSVTVSSGGGGSGGGGSGGGGSDIQMTQTTS SLSASLGDRVTISCRASQDISKYLNWYQQKPDGTVKLLIYHTSRLHSGVP SRFSGSGSGTDYSLTISNLEQEDIATYFCQQGNTLPYTFGGGTKLEITKA
SGGGGSHHHHHH

The domains in the sequence above are in order:
RQR8 marker gene
2A
Signal peptide
aGFP GBP6
Hinge linker (HNG)
CD28 transmembrane
41BB endodomain
CD3Z endodomain
E2A
Signal peptide
aGFP kub4
L3 serine-glycine linker
aCD19FMC63 HL
Hexahistidine tag

Anti-EpCAM_MT110 - Anti-EpCAM CAR co-expressing RQR8 marker gene, with anti-EpCAM MT110 scFv_LH orientation, IgG1 hinge spacer, CD28TM domain and 41BB and CD3Z intracellular domains.

RQR8-2A-aEpCAM_MT110_LH-HNG-CD28TM-41BBZ (SEQ ID N
O: 14)

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLLTCGDVEENPGPMETDTLLLWVLLLWVPGSTGELV MTQSPSSLTVTAGEKVTMSCKSSQSLLNSGNQKNYLTWYQQKPGQPPKLL IYWASTRESGVPDRFTGSGSGTDFTLTISSVQAEDLAVYYCQNDYSYPLT FGAGTKLEIKGGGGSGGGGSGGGGSEVQLLEQSGAELVRPGTSVKISCKA SGYAFTNYWLGWVKQRPGHGLEWIGDIFPGSGNIHYNEKFKGKATLTADK SSSTAYMQLSSLTFEDSAVYFCARLRNWDEPMDYWGQGTTVTVSSSDPAE PKSPDKTHTCP PCPKDPKFWVLVVVGGVLACYSLLVTVAFIIFWVRSKRG RKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQELYNEL
QKDMAEAYSEIGMKGERRRGKGHDGLYQGLSTATKDTYDALHMQALPPR

The domains in the sequence above are in order:
RQR8 marker gene
2A
Signal peptide
aEpCAM MT100 LH
Hinge linker (HNG)
CD28 transmembrane
41BB endodomain
CD3Z endodomain

aP53_421 +BSB_MT110/FMC63 - Anti-p53 CAR co-expressing RQR8 marker gene, with anti-p53 pAb421 scFv LH orientation, IgG1 hinge spacer, CD28TM domain and 41BB and CD3Z intracellular domains; and hexahistidine tagged bispecific binder targeting EpCAM (MT110_LH) and CD19 (FMC63_HL).

RQR8-2A-aP53_421_LH-HNG-CD28TM-41BBZ-E2A-aEpCAM_MT
110_LH-L-aCD19_FMC63_HL-H6 [Also referred to as: a
P53_421 +BSB_MT110/FMC63.](SEQ ID NO: 16)

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLLTCGDVEENPGMETDTLLLWVLLLWVPGSTGDVLM TQTPLTLSVTIGQPASISCKSSQSLLDSDGKTYLNWLLQRPGQSPKRLIY LVSKLDSGVPDRFTGSGSGTDFTLKINRVEAEDLGVYYCWQGTHSPLTFG AGTKLEIKRSGGGGSGGGGSGGGGSQVQLQQSGAELVRSGASVKLSCTAS GFNIKDYYMHWVKQRPEQGLEWIGWIDPENGDTEYAPKFQGKATMTADTS SNTAYLQLSSLASEDTAVYYCNFYGDALDYWGQGTTVTVSSDPAEPKSPD KTHTCP PCPKDPKFWVLVVVGGVLACYSLLVTVAFIIFWVRSKRGRKKLL YIFKQPFMRPVQTTQEEDGCSCRFPEEEEGGCELRVKFSRSADAPYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPRQCTNY ALLKLAGDVESNPGPMYRMQLLSCIALSLALVTNSELVMTQSPSSLTVTA GEKVTMSCKSSQSLLNSGNQKNYLTWYQQKPGQPPKLLIYWASTRESGVP DRFTGSGSGTDFTLTISSVQAEDLAVYYCQNDYSYPLTFGAGTKLEIKGG GGSGGGGSGGGGSEVQLLEQSGAELVRPGTSVKISCKASGYAFTNYWLGW VKQRPGHGLEWIGDIFPGSGNIHYNEKFKGKATLTADKSSSTAYMQLSSL TFEDSAVYFCARLRNWDEPMDYWGQGTTVTVSS GGGGSEVKLQESGPGLV APSQSLSVTCTVSGVSLPDYGVSWIRQPPRKGLEWLGVIWGSETTYYNSA LKSRLTIIKDNSKSQVFLKMNSLQTDDTAIYYCAKHYYYGGSYAMDYWGQ GTSVTVSSGGGGSGGGGSGGGGSDIQMTQTTSSLSASLGDRVTISCRASQ DISKYLNWYQQKPDGTVKLLIYHTSRLHSGVPSRFSGSGSGTDYSLTISN LEQEDIATYFCQQGNTLPYTFGGGTKLEITKASGGGGSHHHHHH

The domains in the sequence above are in order:
RQR8 marker gene
2A
Signal peptide
aP53 421 LH
Hinge linker (HNG)
CD28 transmembrane
41BB endodomain
CD3Z endodomain
E2A
Signal peptide
aEpCAM MT110 LH
L serine-glycine linker
aCD19 FMC63 HL
Hexahistidine tag

In one aspect of the present invention, the cell surface antigen is EpCAM. In one aspect of the present invention, the tumour antigen is p53. In one aspect a binding domain binds a tumour-specific epitope of p53, such as a tumour specific epitope of p53 R175H.

In one aspect of the present invention, the cell surface antigen is EpCAM and the tumour antigen is p53.

Suitably, the chimeric antigen receptor (CAR) may comprise a binding domain which binds a first epitope of p53; and (ii) the bi-specific protein may comprise:
a first binding domain which binds a second epitope of p53; and
a second binding domain which binds EpCAM. Suitably, the first or second epitope of the tumour antigen comprises a tumour-specific mutation such as p53 mutant R175H.

Exemplary sequences and domains for use according to the present invention include:

aP53_421 +BSB_MT110/7B9 - Anti-p53 CAR co-expressing RQR8 marker gene, with anti-p53 pAb421 scFv LH orientation, IgG1 hinge spacer, CD28TM domain and 41BB and CD3Z intracellular domains; and hexahistidine tagged bispecific binder targeting EpCAM (MT110_LH) and mutp53 R175H (7B9_HL).

RQR8-2A-aP53_421_LH-HNG-CD28TM-41BBZ-E2A-aEpCAM_MT
110_LH-LaP53_R175Hmut_7B9_HL-H6 [Also referred to
as: aP53_421 +BSB_MT110/7B9.] (SEQ ID NO:15)

MGTSLLCWMALCLLGADHADACPYSNPSLCSGGGGSELPTQGTFSNVSTN VSPAKPTTTACPYSNPSLCSGGGGSPAPRPPTPAPTIASQPLSLRPEACR PAAGGAVHTRGLDFACDIYIWAPLAGTCGVLLLSLVITLYCNHRNRRRVC KCPRPVVRAEGRGSLLTCGDVEENPGPMETDTLLLWVLLLWVPGSTGDVL MTQTPLTLSVTIGQPASISCKSSQSLLDSDGKTYLNWLLQRPGQSPKRLI YLVSKLDSGVPDRFTGSGSGTDFTLKINRVEAEDLGVYYCWQGTHSPLTF GAGTKLEIKRSGGGGSGGGGSGGGGSQVQLQQSGAELVRSGASVKLSCTA SGFNIKDYYMHWVKQRPEQGLEWIGWIDPENGDTEYAPKFQGKATMTADT SSNTAYLQLSSLASEDTAVYYCNFYGDALDYWGQGTTVTVSSDPAEPKSP DKTHTCP PCPKDPKFWVLVVVGGVLACYSLLVTVAFIIFWVRSKRGRKKL LYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPRQCT NYALLKLAGDVESNPGPMYRMQLLSCIALSLALVTNSELVMTQSPSSLTV TAGEKVTMSCKSSQSLLNSGNQKNYLTWYQQKPGQPPKLLIYWASTRESG VPDRFTGSGSGTDFTLTISSVQAEDLAVYYCQNDYSYPLTFGAGTKLEIK GGGGSGGGGSGGGGSEVQLLEQSGAELVRPGTSVKISCKASGYAFTNYWL GWVKQRPGHGLEWIGDIFPGSGNIHYNEKFKGKATLTADKSSSTAYMQLS SLTFEDSAVYFCARLRNWDEPMDYWGQGTTVTVSS GGGGSEVQLQQSGPE LVKPGASVKISCKTSGYTFTEYTMHWMKQSHGKSLEWIGRINPYSGGTVY NQKFKGKATLTVDKSSSTAYMELRSLTSDDSAVYYCARWGGDYVTGGGTT LTVSSGGGGSGGGGSGGGGSDIVMTQSPSSLSVSGGEKVTMSCKSSQSLL NSGNQKSNLAWYQQKPGQPPKLLIYGASTRESGVPDRFAGSGSGTDFTLT ISSVQAEDLAVYYCQNDHSYPLTFGGGTKLELKRSGGGGSHHHHHH

The domains in the sequence above are in order:
RQR8 marker gene
2A
Signal peptide
aP53 421 LH
Hinge linker (HNG)
CD28 transmembrane
41BB endodomain
CD3Z endodomain
E2A
Signal peptide
aEpCAM MT110 LH
L serine-glycine linker
aP53 R175Hmut 7B9 HL
Hexahistidine tag

An illustrative amino acid sequence for use in the present invention (e.g. a CAR and/or bispecific protein) may comprise a sequence as shown in SEQ ID NO: 1 to 5 or 14 to 16, or a variant thereof with at least 80% (such as at least 85%, at least 90%, at least 95%, at least 97%, at least 99%) identity to any of SEQ ID NO: 1 to 5 or 14 to 16 provided that the variant protein is capable of acting as a chimeric antigen receptor or bi-specific protein. Suitably, an amino acid sequence for use in the present invention (e.g. a CAR and/or bi-specific protein) may comprise one or more domains as shown above from SEQ ID NO: 1 to 5 or 14 to 16 or a variant thereof with at least 80% (such as at least 85%, at least 90%, at least 95%, at least 97%, at least 99%) identity to domains within SEQ ID NO: 1 to 5 or 14 to 16 provided that the variant protein is capable of acting as a chimeric antigen receptor or bi-specific protein.

### Cell Surface Tissue Antigen

As used herein, the term “cell surface tissue antigen″” refers to an antigen which is expressed on the surface of a cell. In other words, at least part of the protein is exposed to the extracellular space.

The cell surface tissue antigen may be a plasma membrane protein which has at least part of a domain exposed to the extracellular space or the exoplasmic surface of the plasma membrane. The cell surface tissue antigen may be an integral (or intrinsic) membrane protein. Integral membrane proteins are permanently attached to the membrane and have one or more domains which are embedded in the phospholipid bilayer. Typically, integral membrane proteins have residues with hydrophobic side chains that interact with fatty acyl groups of the membrane phospholipids anchoring the protein to the membrane. Examples of integral membrane proteins include transporters, channels, receptors and cell adhesion proteins.

The cell surface tissue antigen may be a transmembrane protein. Transmembrane proteins span the lipid bilayer. Transmembrane proteins may be single or multi-pass membrane proteins. For example, the transmembrane protein may be a member of the immunoglobulin superfamily. The cell surface protein may be an integral monotropic protein. Integral monotropic proteins are associated with one side of the lipid bilayer and do not span the lipid bilayer.

The cell surface protein may be a peripheral (or extrinsic) membrane protein. Peripheral membrane proteins do not interact with the hydrophobic core of the phospholipid bilayer. Peripheral membrane proteins are typically bound to the membrane indirectly by interactions with integral membrane proteins or directly by interactions with lipid polar head groups. Peripheral proteins may be localized to the outer (exoplasmic) surface of the plasma membrane. The cell surface protein may be a peripheral exoplasmic membrane protein.

The cell surface tissue antigen may be anchored to the plasma membrane e.g. covalently attached to lipids embedded within the cell membrane (such as via a glycosylphosphatidylinositol (GPI) anchor).

The cell surface membrane protein may be a GPI-anchored protein.

Various methods exist for determining the subcellular localisation of proteins are well known in the art and include for example: electron microscopy; confocal microscopy using surface biotinylation or co-localisation with known membrane proteins; immuno-fluorescence; and flow cytometry with fluorescently tagged antibodies.

The cell surface tissue antigen is expressed in the tumour microenvironment. Suitably, the cell surface tissue antigen may be expressed on the tumour cell. Suitably, the cell surface tissue antigen may be expressed by cells other than the tumour cell in the tumour microenvironment. For example, the cell surface tissue antigen may be expressed on cells in the tumour microenvironment which are themselves not tumour cells. For example, the cell surface tissue antigen may be expressed on blood vessels (such as tumour associated endothelial cells), stromal cells, immune cells, tumour associated macrophages, fibroblasts in the tumour microenvironment.

The cell surface tissue antigen may be expressed by tumour cells and by non-cancerous cells of the same lineage.

Cell surface expression may be determined using any method known in the art, for example by flow cytometry using an appropriate isotype matched control which helps to differentiate nonspecific background signal from specific antibody signal.

The cell surface antigen enables the bi-specific protein to accumulate in the tumour microenvironment. The cell surface antigen may be any antigen useful in targeting the treatment of a solid cancer.

The cell surface antigen may be an epithelial antigen. For example, the cell surface antigen may be a pan-epithelial antigen, pan-glial antigen, pan-lung antigen, pan-bowel mucosa antigen, pan breast epithelium antigen, pan ovarian antigen.

Table 8 below lists exemplary epithelial antigens which may be used in the present invention. Suitably, the bi-specific protein acceding to any aspect of the present invention may comprise a binding domain which binds to any of the epithelial antigens listed in Table 9 below.

TABLE 9

Exemplary epithelial cell surface antigens

| | |
|---|---|
| Target | Tissue expression |
| CD326 (EpCAM) | Pan epithelial cell marker |
| EGFR (ErbB-1, HER1) | Lung. Breast. CNS. Prostate. Gastric. Placenta. Colorectal. Pancreatic. Neuroendocrine. Ovarian. |
| CD227 (MUC1, EMA) | Breast. Oesophageal. Gastric. Colorectal. Pancreatic. Prostate. Lung. Ovarian. |
| GPA33 | Colorectal. |
| TACSTD2 (GA733-1, TROP-2, EGP-1) | Epithelial cell marker |
| EMP2 | Brain. Liver. Prostate. Ovarian. Neuroendocrine. Heart. Lung. Gastric. Pancreatic. |
| CD324 (E-cadherin, CHD1) | Pan epithelial marker |
| P-cadherin (CDH3) | Pan stratified epithelial marker |
| CD325 (N-cadherin, CDH2) | Neuroendocrine. Pancreatic. Liver. Endometrial. Uterus. Ovarian. Gastric. CNS. |
| CD66e (CEA) | Gastric. Urogenital. Lung. Colorectal. Oesophageal. |
| CD44; CD44s, CD44v6, CD44v4-10, CD44v8-10 | CNS. Neuroendocrine. Ovarian. Testis. Kidney. Liver. Heart. Lung. Colorectral. Gastric. Pancreatic. |
| Integrins, including CD49f (integrin alpha-6) | CNS. Neuroendocrine. Ovarian. Testis. Kidney. Liver. Heart. Lung. Colorectral. Gastric. Pancreatic. |
| CD340 (ErbB-2, HER2) | Breast. Ovarian. Gastric. Colorectal. Pancreatic. Endometrial. Lung. Kidney. Liver. Skin. |
| HER3 (ErbB-3) | CNS. Ovarian. Testis. Kidney. Liver. Heart. Breast. Lung. Colorectal. Endocrine. Gastric. |
| HER4 (ErbB-4) | CNS. Testis. Kidney. Heart. Lung. Gastric. Urogenital. |
| CD10 | Neuroendocrine. Endometrial. Urogenital. Liver. Kidney. Brain. Breast. Colorectal. Gastric. Ovarian. Placenta. Prostate. |
| ENaC-A (SCNN1A) | Kidney. Lung. Salivary glands. Skin. Colon. Placenta. |
| ENaC-B (SCNN1B) | Kidney. Lung. Salivary glands. Skin. Colon. Placenta. |
| ENaC-G (SCNN1G) | Kidney. Lung. Salivary glands. Skin. Colon. Placenta. |
| ENaC-D (SCNN1D) | Kidney. Lung. Neuroendocrine. CNS. |
| CD118 (LIFR) | Liver. Placenta. CNS. Skin. Lung. Gastric. Prostate. Ovarian. Neuroendocrine. Testis. Pancreatic. Colorectal. Oesophageal. Heart. |
| PSMA | Prostate. Urogential. Gastrointestinal. Kidney. Liver. |
| PSCA | Prostate. Urogential. Gastrointestinal. Kidney. Liver. Oesophageal. Skin. Placenta. |
| STEAP1 | Prostate. Lung. Breast. Pancreatic. Urogenital. Ovarian. Gastric. Bladder. Colorectal. |
| CD331 (FGFR1) | Gastric. CNS. Brain. Kidney. Placenta. Neuroendocrine. Lung. Colorectal. Liver. Pancreatic. Urogenital. |
| CD332 (FGFR2) | Gastric. CNS. Brain. Kidney. Placenta. Neuroendocrine. Lung. Colorectal. Liver. Pancreatic. Urogenital. |
| CD333 (FGFR3) | Gastric. CNS. Brain. Kidney. Placenta. Neuroendocrine. Lung. Colorectal. Liver. Pancreatic. Urogenital. |
| CD334 (FGFR4) | Gastric. CNS. Brain. Kidney. Placenta. Neuroendocrine. Lung. Colorectal. Liver. Pancreatic. Urogenital. Breast. Cervix. |
| CD166 (ALCAM) | Neuroendocrine. CNS. Lung. Bladder. Pancreatic. Stomach. Urogenital. Kidney. Testis. Prostate. Breast. Endometrial. Cervix. |
| Mesothelin | Lung. Ovarian. Pancreatic.. |
| EPHA2 | CNS. Lung. Neuroendocrine. Colon. Urogenital. Uterus Testis. Prostate. Gastric. Oesophageal. Endometrial. Cervix. Pancreatic. Kidney. Liver. |
| CD24 | Lung. Breast. CNS. Neuroendocrine. Kidney. Colorectal. Bladder. Oesophageal. Ovarian. Prostate. |
| Claudin-7 | Kidney. Lung. Colorectal. Pancreas. Urogenital. Liver. Bladder. Neuroendocrine. |

In some aspects, the cell surface tissue antigen may be for example: cluster of differentiation (CD) proteins; cell adhesion or cell junction proteins; G protein coupled receptors; solute carrier family members and tetraspanins. The cell surface tissue antigen may be a member of the immunoglobulin superfamily.

The cell surface antigen may be a cluster of differentiation (CD) protein. The CD nomenclature has been used for the identification and investigation of cell surface molecules providing targets for immunophenotyping of cells.

For example, the cell surface antigen may be selected from:
CD19, CD20, CD45, CD38 or CD22 (typically expressed by B cells);
CD33 (typically expressed by cells of myeloid lineage);
CD2 or CD5 (typically expressed by T cells);
CD34 or CD117 (typically expressed by stem cells);
CD69 (typically expressed by activated cells); and
CD31 (typically expressed by endothelial cells, platelets, macrophages, granulocytes and lymphocytes).

In one aspect, the cell surface antigen is CD326 (EpCAM).Epithelial call adhesion molecule is a transmembrane glycoprotein mediating Ca2+ -independent homotypic cell-cell adhesion in epithelia. EpCAM also plays a role in cell signalling, migration, proliferation and differentiation and is expressed in epithelia and epithelial-derived neoplasms.

The cell surface tissue antigen may be CD19. CD19 is a transmembrane glycoprotein belonging to the immunoglobulin superfamily and is widely expressed during all phases of B cell development until terminal differentiation into plasma cells. CD19 is also expressed on the surface of neoplastic B cells, for example it is expressed at normal to high levels in most acute lymphoblastic leukaemia’s (ALL), chronic lymphocytic leukaemias (CLL) and B cell lymphomas. Many binding domains which recognise and bind to cell surface tissue antigens are available. For example, Naddafi and Davami Int J Mol Cell Med. 2015 Summer; 4(3): 143-151 report anti-CD19 monoclonal antibodies which are being used in new approaches to lymphoma therapy.

For example, Blinatumomab is a bi-specific T-cell engager (scFv) which comprises a binding domain for CD19 and a binding domain for CD3; SAR3419 is an antibody-drug conjugate; MOR-208 is an Fc engineered antibody and MEDI-551 is a glycol-engineered antibody.

The cell surface tissue antigen may be CD33. CD33 is a transmembrane receptor expressed on myeloid lineage cells. It binds sialic acids and is a member of the SIGLEC family of lectins, within the immunoglobulin superfamily. CD33 is stimulated by binding of a molecule which comprises sialic acid residues. Binding of the sialic acid residue results in phosphorylation of the immunoreceptor-tyrosine-based inhibition motif (ITIM) on the cytosolic portion of CD33 which acts as a docking site for Src homology (SH2) domain containing proteins such as SHP phosphatases. This signalling cascade through CD33 inhibits phagocytosis. Binding domains which recognise and bind to CD33 are known in the art. For example, vadastuximab talirine and gemtuzumab ozogamicin are antibody-drug conjugates which target CD33 for the treatment of acute myeloid leukaemia.

In some aspects, the cell surface tissue antigen may be specific for the tumour. As used herein “specific for the tumour” means that the antigen is not expressed or is expressed at a lower amount in non-cancerous cells of the same lineage.

Suitably, the cell surface tissue antigen may be expressed at a level which is at least 25%, at least 30%, at least 40%, at least 50%, at least 60%, at least 70%, at least 80%, at least 90%, at least 95% lower in a non-cancerous cell of the same lineage when compared to the tumour.

The level of expression may be calculated as a percentage of cells which are positive for the cell surface tissue antigen, for example by flow cytometry. A comparison may be made between a population of cells taken from the tumour and a population of cells from a corresponding non-cancerous tissue or a population of non-cancerous cells of the same lineage.

### NUCLEIC ACIDS

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector.

As used herein, the terms “polynucleotide” and “nucleic acid” are intended to be synonymous with each other. The nucleic acid sequence may be any suitable type of nucleotide sequence, such as a synthetic RNA/DNA sequence, a cDNA sequence or a partial genomic DNA sequence.

The term “polypeptide” as used herein is used in the normal sense to mean a series of residues, typically L-amino acids, connected one to the other typically by peptide bonds between the α-amino and carboxyl groups of adjacent amino acids. The term is synonymous with “protein”.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

The present invention provides a polynucleotide which encodes a CAR according to the present invention. The present invention provides a polynucleotide which encodes a bi-specific protein according to the present invention. Suitably, the polynucleotide may encode both a CAR and a bi-specific protein according to the present invention.

Nucleic acids encoding CARs and/or bi-specific proteins according to the present invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The polynucleotide may be in isolated or recombinant form. It may be incorporated into a vector and the vector may be incorporated into a host cell. Such vectors and suitable hosts form yet further aspects of the present invention.

The polynucleotide which encodes the CAR and/or bi-specific protein according to the present invention may be codon optimised. Different cells differ in their usage of particular codons. This codon bias corresponds to a bias in the relative abundance of particular tRNAs in the cell type.

By altering the codons in the sequence so that they are tailored to match with the relative abundance of corresponding tRNAs, it is possible to increase expression. Suitably the polynucleotide may be codon optimised for expression in a murine model of disease. Suitably, the polynucleotide may be codon optimised for expression in a human subject.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation may also involve the removal of mRNA instability motifs and cryptic splice sites.

Suitably, the polynucleotide may comprise a nucleic acid sequence which enables both a nucleic acid sequence encoding a CAR and a nucleic acid sequence encoding a bi-specific protein be expressed from the same mRNA transcript.

For example, the polynucleotide may comprise an internal ribosome entry site (IRES) between the nucleic acid sequences which encode the CAR and the bi-specific protein. An IRES is a nucleotide sequence that allows for translation initiation in the middle of a mRNA sequence.

The internal self-cleaving sequence may be any sequence which enables the polypeptide comprising the CAR and the polypeptide comprising the bi-specific protein to become separated.

The cleavage site may be self-cleaving, such that when the polypeptide is produced, it is immediately cleaved into individual peptides without the need for any external cleavage activity. The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide, various models have been proposed for to account for the “cleavage” activity: proteolysis by a host-cell proteinase, autoproteolysis or a translational effect (Donnelly et al., (2001) J. Gen. Virol. 82:1027-1041, incorporated herein by reference). The exact mechanism of such “cleavage” is not important for the purposes of the present invention, as long as the cleavage site, when positioned between nucleic acid sequences which encode proteins, causes the proteins to be expressed as separate entities.

The self-cleaving peptide may be a 2A self-cleaving peptide from an aphtho- or a cardiovirus. The present invention provides a nucleic acid construct which comprises a nucleic acid sequence which encodes a CAR and/or a bi-specific protein according to the present invention.

### Vector

The present invention also provides a vector comprising a nucleotide sequence encoding a CAR and/or bi-specific protein as described herein.

The present invention also provides a vector comprising a nucleic acid construct encoding a CAR and/or bi-specific protein as described herein.

Suitably, the vector may comprise a nucleotide sequence encoding a CAR of the present invention.

Suitably, the vector may comprise a nucleotide sequence encoding a bi-specific protein of the present invention.

In one aspect, there is provided a kit of vectors which comprises one or more nucleic acid sequence(s) of the invention such as a nucleic acid encoding a CAR and a nucleic acid encoding a bi-specific protein of the present invention.

The term “vector” as used herein includes an expression vector, i.e., a construct enabling expression of a CAR and/or bi-specific protein according to the present invention.

Suitably the expression vector enables expression of a CAR and/or bi-specific protein according to the present invention.

In some embodiments, the vector is a cloning vector.

Suitable vectors may include, but are not limited to, plasmids, viral vectors, transposons, nucleic acid complexed with polypeptide or immobilised onto a solid phase particle.

Viral delivery systems include but are not limited to adenovirus vector, an adeno-associated viral (AAV) vector, a herpes viral vector, retroviral vector, lentiviral vector, baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al., (“Retroviruses” 1997 Cold Spring Harbour Laboratory Press Eds: JM Coffin, SM Hughes, HE Varmus pp 758-763), incorporated herein by reference.

Lentiviruses also belong to the retrovirus family, but they can infect both dividing and non-dividing cells (Lewis et al., (1992) EMBO J. 3053-3058), incorporated herein by reference.

The vector may be capable of transferring a polynucleotide the invention to a cell, for example a host cell as defined herein. The vector should ideally be capable of sustained high-level expression in host cells, so that the VH and/or VL domain are suitably expressed in the host cell.

The vector may be a retroviral vector. The vector may be based on or derivable from the MP71 vector backbone. The vector may lack a full-length or truncated version of the Woodchuck Hepatitis Response Element (WPRE).

For efficient infection of human cells, viral particles may be packaged with amphotropic envelopes or gibbon ape leukemia virus envelopes.

### Cell

The present invention further provides an engineered cell comprising a CAR and/or bi-specific protein according to the present invention. In one aspect, the engineered cell may comprise a polynucleotide or vector which encodes a CAR according to the present invention. In one aspect, the engineered cell may comprise a polynucleotide or vector which encodes a bispecific protein according to the present invention.

The engineered cell may be any cell which can be used to express and produce a CAR and/or bi-specific protein according to the present invention.

Suitably the cell may be an immune effector cell.

“Immune effector cell” as used herein is a cell which responds to a stimulus and effects a change i.e. the cell carries out a response to the stimulus. Immune effector cells may include alpha/beta T cells, gamma/delta T cells, Natural killer (NK) cells and macrophages.

Suitably, the cell may be an alpha/beta T cell.

Suitably, the cell may be a gamma/delta T cell.

Suitably, the cell may be a T cell, such as a cytolytic T cell e.g. a CD8+ T cell.

Suitably, the cell may be an NK cell, such as a cytolytic NK cell.

Suitably, the cell may be a macrophage.

In one aspect, the cell may be isolated from blood obtained from the subject. Suitably, the cell may be isolated from peripheral blood mononuclear cells (PBMCs) obtained from the subject.

In one aspect, the cell may be a stem cell.

In another aspect, the cell may be a progenitor cell.

As used herein, the term “stem cell” means an undifferentiated cell which is capable of indefinitely giving rise to more stem cells of the same type, and from which other, specialised cells may arise by differentiation. Stem cells are multipotent. Stem cells may be for example, embryonic stem cells or adult stem cells.

As used herein, the term “progenitor cell” means a cell which is able to differentiate to form one or more types of cells but has limited self-renewal in vitro.

Suitably, the cell may be capable of being differentiated into a T cell.

Suitably, the cell may be capable of being differentiated into an NK cell.

Suitably, the cell may be capable of being differentiated into a macrophage.

Suitably, the cell may be an embryonic stem cell (ESC). Suitably, the cell is a haematopoietic stem cell or haematopoietic progenitor cell. Suitably, the cell is an induced pluripotent stem cell (iPSC). Suitably, the cell may be obtained from umbilical cord blood. Suitably, the cell may be obtained from adult peripheral blood.

In some aspects, hematopoietic stem and progenitor cell (HSPCs) may be obtained from umbilical cord blood. Cord blood can be harvested according to techniques known in the art (e.g., U.S. Pat. Nos. 7,147,626 and 7,131,958 which are incorporated herein by reference).

In one aspect, HSPCs may be obtained from pluripotent stem cell sources, e.g., induced pluripotent stem cells (iPSCs) and embryonic stem cells (ESCs).

As used herein, the term “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(-) cells includes haematopoietic stem cells and hematopoietic progenitor cells.

HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton’s jelly, placenta, fetal blood, fetal liver, or fetal spleen. In particular embodiments, harvesting a sufficient quantity of HSPCs for use in therapeutic applications may require mobilizing the stem and progenitor cells in the subject.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state.

As used herein, the term “programming” refers to a method of decreasing the potency of a cell or differentiating the cell to a more differentiated state.

Suitably the cell is matched or is autologous to the subject. The cell may be generated ex vivo either from a patient’s own peripheral blood (1st party), or in the setting of a haematopoietic stem cell transplant from donor peripheral blood (2nd party), or peripheral blood from an unconnected donor (3rd party).

Suitably the cell may be autologous to the subject.

In some aspects, the cell may be derived from ex vivo differentiation of inducible progenitor cells or embryonic progenitor cells to the immune cell. In these instances, cells are generated by introducing DNA or RNA coding for the CAR of the present invention by one of any means including transduction with a viral vector, transfection with DNA or RNA.

### Compositions

The present invention also provides a composition comprising a cell according to the invention, such as an engineered immune effector cell. Suitably, the composition may comprise a population of cells according to the present invention. Suitably, the composition may comprise a bi-specific protein according to the present invention.

Suitably the present invention provides a composition comprising an engineered cell comprising a CAR according to the present invention. Suitably the composition may comprise a population of engineered cells comprising a CAR according to the present invention. Suitably the present invention provides a composition comprising an engineered cell which expresses a bi-specific protein according to the present invention. Suitably the composition may comprise a population of engineered cells which express a bi-specific protein according to the present invention.

In some embodiments, the composition is a pharmaceutical composition. Such pharmaceutical composition may comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents.

The pharmaceutical compositions typically should be sterile and stable under the conditions of manufacture and storage. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. A pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers which are non-toxic to the subjects at the dosages and concentrations employed. Preferably, such a composition can further comprise a pharmaceutically acceptable carrier or excipient for use in the treatment of disease that that is compatible with a given method and/or site of administration, for instance for parenteral (e.g. sub-cutaneous, intradermal, or intravenous injection) or intrathecal administration.

Wherein the pharmaceutical composition comprises a cell according to the invention, the composition may be produced using current good manufacturing practices (cGMP).

Suitably the pharmaceutical composition comprising a cell according to the present invention may comprise an organic solvent, such as but not limited to, methyl acetate, dimethyl sulfoxide (DMSO), N,N-dimethylformamide (DMF), dimethoxyethane (DME), and dimethylacetamide, including mixtures or combinations thereof.

Suitably the pharmaceutical composition comprising a cell according to the present invention is endotoxin free.

### Method of Treatment/Uses

The present invention provides a method for treating and/or preventing a disease which comprises the step of administering a cell of the present invention or obtainable (e.g. obtained) by a method according to the present invention to a subject.

The present invention provides a method for treating and/or preventing a disease which comprises the step of administering a pharmaceutical composition of the present invention or obtainable (e.g. obtained) by a method according to the present invention to a subject.

The present invention also provides a cell of the present invention or obtainable (e.g. obtained) by a method according to the present invention for use in treating and/or preventing a disease. The present invention also provides a pharmaceutical composition of the present invention for use in treating and/or preventing a disease.

The invention also relates to the use of a cell according to the present invention in the manufacture of a medicament for treating and/or preventing a disease.

Preferably, the present methods of treatment relate to the administration of a pharmaceutical composition of the present invention to a subject.

The term “treat/treatment/treating” refers to administering a cell or pharmaceutical composition to a subject having an existing disease or condition in order to lessen, reduce or improve at least one symptom associated with the disease and/or to slow down, reduce or block the progression of the disease.

Reference to “prevention”/“preventing” (or prophylaxis) as used herein refers to delaying or preventing the onset of the symptoms of the disease. Prevention may be absolute (such that no disease occurs) or may be effective only in some individuals or for a limited amount of time.

In a preferred embodiment of the present invention, the subject of any of the methods described herein is a mammal, preferably a cat, dog, horse, donkey, sheep, pig, goat, cow, mouse, rat, rabbit or guinea pig. Preferably the subject is a human.

The administration of a pharmaceutical composition of the invention can be accomplished using any of a variety of routes that make the active ingredient bioavailable. For example, a cell or pharmaceutical composition according to the invention may be administered intravenously, intrathecally, by oral and parenteral routes, intranasally, intraperitoneally, subcutaneously, transcutaneously or intramuscularly.

Suitably, the cell according to the present invention or the pharmaceutical composition according to the invention may be administered intravenously.

Suitably, the cell according to the present invention or the pharmaceutical composition according to the present invention is administered intrathecally.

Typically, a physician will determine the actual dosage that is most suitable for an individual subject and it will vary with the age, weight and response of the particular patient. The dosage is such that it is sufficient to reduce and/or prevent disease symptoms.

Those skilled in the art will appreciate, for example, that route of delivery (e.g., oral vs intravenous vs subcutaneous, etc.) may impact dose amount and/or required dose amount may impact route of delivery. For example, where particularly high concentrations of an agent within a particular site or location are of interest, focused delivery may be desired and/or useful. Other factors to be considered when optimizing routes and/or dosing schedule for a given therapeutic regimen may include, for example, the disease being treated (e.g., type or stage, etc.), the clinical condition of a subject (e.g., age, overall health, etc.), the presence or absence of combination therapy, and other factors known to medical practitioners.

The dosage is such that it is sufficient to stabilise or improve symptoms of the disease.

The present invention also provides a method for treating and/or preventing a disease, which comprises the step of administering a pharmaceutical composition comprising an engineered cell e.g. a cell which has been engineered to express a CAR according to the present invention to a subject.

Suitably, the present invention also provides a method for treating and/or preventing a disease, which comprises the step of administering a cell according to the invention or obtainable (e.g. obtained) by a method according to the present invention to a subject.

In one aspect, the method may comprise the following steps:
(i) isolation of a cell-containing sample from a subject;
(ii) introducing a) a polynucleotide encoding a CAR comprising a binding domain which binds a first epitope of a tumour antigen; and b) a polynucleotide which encodes a bispecific protein which comprises a first binding domain which binds a second epitope of said tumour antigen; and a second binding domain which binds a cell surface antigen; to said isolated cell from (i) and
(iii) administering the cells from (ii) to the subject.

In another aspect, the method may comprise:
administering a bi-specific protein which comprises: a first binding domain which binds a second epitope of said tumour antigen; and a second binding domain which binds a cell surface antigen; to a subject,
wherein said subject comprises engineered cells comprising a nucleic acid sequence encoding a CAR comprising a binding domain which binds a first epitope of a tumour antigen.

In another aspect, the method may comprise the following steps:
(i) isolation of a cell-containing sample from a subject;
(ii) introducing a polynucleotide encoding a CAR comprising a binding domain which binds a first epitope of a tumour antigen to said isolated cell from (i);
(iii) administering the cells from (ii) to the subject; and
(iv) administering a polynucleotide which encodes a bi-specific protein which comprises:
   a first binding domain which binds a second epitope of said tumour antigen; and a second binding domain which binds a cell surface antigen; to the subject comprising the engineered CAR cells.

Suitably, the cells from (ii) may be expanded in vitro before administration to the subject.

### Disease

The disease may be, for example, a cancer.

Suitably, the disease to be treated and/or prevented by the methods and uses of the present invention may be a cancer.

Suitably, the disease to be treated and/or prevented by the methods and uses of the present invention may be a haematological malignancy.

As used herein, “haematological malignancy” refers to a cancer which affects the blood and lymph system and includes leukaemia, lymphoma, myeloma and related blood disorders. Suitably, the disease to be treated and/or prevented by the methods and uses of the present invention may be a malignant solid tumour. Solid tumours include sarcomas, carcinomas and lymphomas.

The disease to be treated and/or prevented may be selected from those listed in Tables 1-4 above.

The disease to be treated and/or prevented may be associated with a phosphoantigen.

For example, the phosphoantigen may be selected from: KRAS (for example wherein the phosphor residue is S181), BRAF (for example, wherein the phosphor residue is T599 and/or S602), Aurora A (for example wherein the phosphor residue is T288 and/or T287) and ERG (for example wherein the phosphor residue is S96 and/or S215.

The disease to be treated and/or prevented may be associated with a fusion protein.

For example, the fusion protein may be selected from: AML4-ALK, CCD6-RET and NCOA4-RET.

The disease to be treated and/or prevented may be associated with a point mutation.

For example, the point mutation may be in TP53 (for example, wherein the point mutation is selected from: R175H, G245S, R248Q, R248W, R249S, R273H, R273C, R282W and/or Y220C), KRAS (wherein the point mutation is selected from: G12C, G12R, G12S, G12A, G12D, G12V, G13D, G13C, G13V, Q61H, Q61R and/or A146T) or BRAF (wherein the point mutation is selected from: V600E, G469A, G469V, K601E, D594N, D594G and/or N581S).

The disease to be treated and/or prevented may be associated with a tumour antigen selected from: PRAME, Survivin, WT1 and telomerase.

### Method

The present invention also provides a method for producing a cell, which method comprises introducing into a cell in vitro or ex vivo, a polynucleotide encoding a CAR as defined herein.

The present invention also provides a method for producing a cell, which method comprises introducing into a cell in vitro or ex vivo, a polynucleotide encoding a bi-specific protein as defined herein. Suitably, the polynucleotides encoding the CAR and the bi-specific protein may be introduced into the same cell. One polynucleotide may encode both the CAR and the bispecific protein.

Suitably, the method may comprise introducing into a cell in vitro or ex vivo, a nucleic acid construct encoding a CAR as defined herein. Suitably, the method may comprise introducing into a cell in vitro or ex vivo, a nucleic acid construct encoding a bi-specific protein as defined herein. Suitably, the nucleic acid constructs encoding the CAR and the bi-specific protein may be introduced to the same cell. One nucleic acid construct may encode both the CAR and the bi-specific protein.

Suitably, the method may comprise introducing into a cell in vitro or ex vivo, a vector which comprises a polynucleotide encoding a CAR as defined herein. Suitably, the method may comprise introducing into a cell in vitro or ex vivo, a vector which comprises a polynucleotide encoding a bi-specific protein as defined herein. Suitably the vectors for encoding the CAR and the bi-specific protein may be introduced to the same cell. One vector may encode both the CAR and the bi-specific protein.

Suitably, the method may further comprise incubating the cell under conditions permitting expression of the CAR molecule and/or bi-specific protein of the present invention. Optionally, the method may further comprise a step of purifying the engineered cells.

Suitably, the cell may be an immune effector cell.

Suitably, the cell may be a cytolytic cell.

Suitably, the cell may be a T cell.

Suitably, the cell may be an NK cell.

In one aspect, the cell may be a stem cell.

Suitably, in the method according to the invention, a nucleic acid encoding a CAR and/or a bispecific protein as defined herein may be introduced into the stem cell and the stem cell is then differentiated into a T cell. Suitably, in the method according to the invention, a nucleic acid encoding a CAR as defined herein may be introduced into the stem cell and the stem cell is then differentiated into an NK cell.

Suitably, the stem cell may have the ability to differentiate into a T cell.

Suitably, the stem cell may have the ability to differentiate into an NK cell.

Suitably, the cell may be an embryonic stem cell (ESC). Suitably, the cell may be obtained from umbilical cord blood. Suitably, the cell may be obtained from adult peripheral blood. Suitably, the cell is a haematopoietic stem and progenitor cell (HSPC). Suitably, the cell is an induced pluripotent stem cell (iPSC).

In another aspect, the cell is a progenitor cell. Suitably the progenitor cell has the ability to differentiate into a T cell. Suitably, the progenitor cell has the ability to differentiate into an NK cell.

In another aspect, the invention provides a method for producing an engineered cell comprising a CAR according to the present invention. In another aspect, the invention provides a method for producing an engineered cell comprising a polynucleotide which encodes a bi-specific protein according to the present invention.

Suitably, the method may comprise introducing into a cell in vitro or ex vivo a polynucleotide encoding a CAR and/or a bi-specific protein according to the present invention.

Suitably, the CAR and the bi-specific protein may be provided by the same polynucleotide. Suitably the CAR and the bi-specific protein may be provided as separate polynucleotides. Suitably, the separate polypeptides may be introduced separately, sequentially or simultaneously into the cell. Wherein the polypeptides are introduced separately or sequentially, suitably the polynucleotide encoding the CAR may be introduced first. Wherein the polypeptides are introduced separately or sequentially, suitably the polynucleotide encoding the bi-specific protein may be introduced first.

Suitably, the method further may comprise incubating the cell under conditions causing expression the CAR molecule and/or bi-specific protein of the present invention. Optionally, the method may further comprise a step of purifying the engineered cells.

In one aspect, the invention provides a method for producing an engineered cell, which method comprises introducing into a cell in vitro or ex vivo a polynucleotide encoding a CAR and differentiating the cell into a T cell. Suitably, the method may further comprise incubating the cell under conditions causing expression of the CAR molecule of the present invention. Optionally, the method may further comprise a step of purifying the engineered cells comprising the CAR according to the invention.

Suitably, in one aspect the cell may be differentiated into a T cell before the one or more polynucleotide(s) encoding the CAR are introduced into the cell.

Purification of the engineered cell may be achieved by any method known in the art. Suitably, the engineered cell may be purified using fluorescence-activated cell sorting (FACS) or immunomagnetic isolation (i.e. using antibodies attached to magnetic nanoparticles or beads) using positive and/or negative selection of cell populations.

Suitably, purification of the engineered cell may be performed using the expression of the CAR as defined herein.

The present invention also provides a method for lysing a tumour cell which releases proteins into the tumour microenvironment, which method comprises introducing to a cell
1) a polynucleotide encoding a CAR which comprises a binding domain which binds a first epitope of a tumour antigen; and
2) a polynucleotide which encodes a bi-specific protein which comprises a first binding domain which binds a second epitope of said tumour antigen; and a second binding domain which binds an epitope of a cell surface tissue antigen.

### Use

The present invention also provides a pharmaceutical composition or cell (e.g. a population of cells such as engineered cells) according to the invention or obtainable (e.g. obtained) by a method according to the present invention for use in treating disease. The pharmaceutical composition or cell(s) (such as engineered cell) may be any as defined above.

The present invention also relates to the use of a cell or population of cells according to the present invention or obtainable (e.g. obtained) by a method according to the present invention as defined above in the manufacture of a medicament for the treatment of a disease.

The present invention also provides a bi-specific protein according to the invention or obtainable (e.g. obtained) by a method according to the present invention for use in treating disease. The bi-specific protein may be any as defined above.

### Car System

The present invention provides a CAR system comprising;
(i) a receptor component comprising a binding domain which binds a first epitope of a tumour antigen, a transmembrane domain and a signaling domain; and
ii) a bi-specific protein which comprises:
   a first binding domain which binds a second epitope of said tumour antigen; and
   a second binding domain which binds an epitope of a cell surface tissue antigen.

The system may comprise an immune effector cell which comprises or expresses the receptor component. For example, the system may comprise an alpha-beta T cell, a NK cell, a gamma-delta T cell, a cytokine induced killer cell or a macrophage which comprises or expresses the receptor component.

The system may comprise an immune effector cell which expresses the bi-specific protein. For example, the system may comprise an alpha-beta T cell, a NK cell, a gamma-delta T cell, a cytokine induced killer cell or a macrophage which expresses the bi-specific protein.

Suitably, the receptor component and the bi-specific protein may be expressed by the same cell such as an immune effector cell.

In some aspects, the system comprises a tumour microenvironment. Suitably, the bi-specific protein may administered to said system. In other words, the bi-specific protein is introduced to the tumour microenvironment. For example, the bi-specific protein is not produced in the tumour microenvironment but is introduced to the tumour microenvironment, by intratumoural injection.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

It is noted that embodiments of the invention as described herein may be combined.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1 - EXOAMP In Vitro Targeting of CD33 Positive and Intracellular eGFP Expressing Cells

As proof of concept, the inventors have conveniently used eGFP as a tumour antigen. CD19 or CD33 was used as tissue-specific antigen. Bi-specific proteins were generated which recognize CD19/eGFP or CD33/eGFP. A CAR was generated which independently recognized eGFP.

In this example the results show that the EXOAMP GFP targeting CAR lyses double positive CD33/eGFP expressing targets and spares single positive CD33 expressing targets.

The HL-60 cell line is a human leukaemia cell line derived from peripheral blood from a patient with acute promyelocytic leuakaemia (PML) is CD19-negative and BCR-ABL negative.

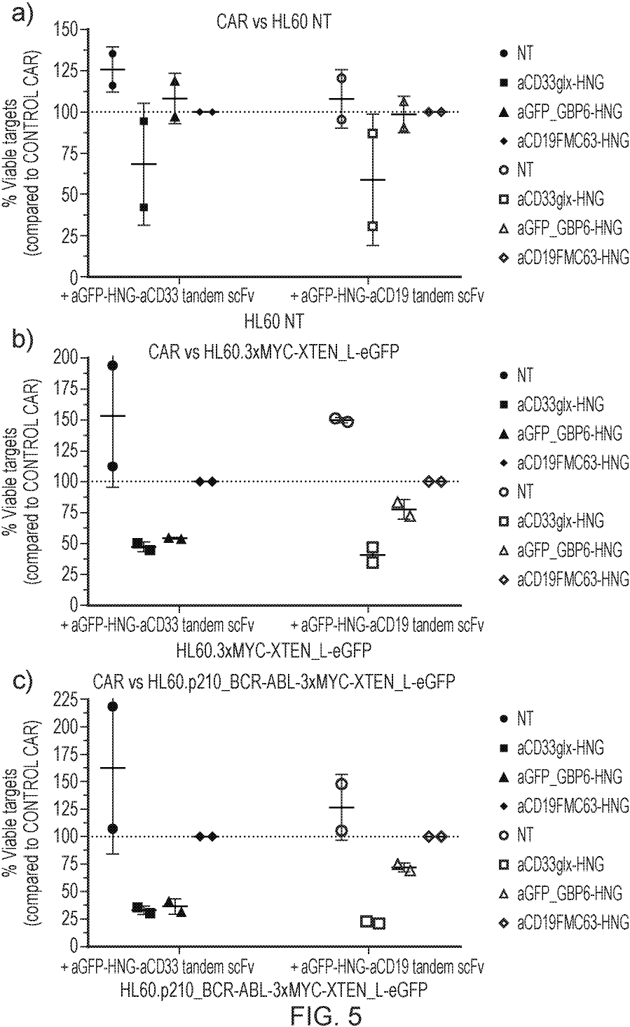

Effector T-cells comprising:
anti-CD33 CAR (αCD33glx-HNG);
anti-GFP CAR (αGFP_GBP6-HNG);
CD19-CAR (αCD19FMC63) - this is the negative control; or
non-transduced (NT);
co-expressing either, a bispecific binder against GFP/CD33 (αGFP-HNG-αCD33 tandem scFv) [see left hand side of FIG. 5 a), b) and c)] or an irrelevant bispecific binder against GFP/CD19 (αGFP-HNG-αCD19) [see right hand side of FIG. 5a), b) and c)] were produced.

The effector T cells were co-cultured at a 1:1 effector to target ratio against:
CD33 expressing HL60 cells - see FIG. 5 a);
HL60 cells expressing intracellular eGFP protein (3xMYC-XTEN_L-eGFP) - see FIG. 5 b); and HL60 cells expressing an intracellular chimeric GFP/BCR-ABL protein (p210.p210_BCR-ABL-3xMYC-XTEN_L-eGFP) - see FIG. 5 c).

The data in FIG. 5 show that the EXOAMP GFP targeting CAR lyses double positive CD33/eGFP expressing targets and spares single positive CD33 expressing targets, as shown by comparison of the triangles in FIG. 5 a), b) and c).

An exemplary sequence used in this example is: p210_BCR-ABL-3xMYC-XTEN_L-eGFP (SEQ ID NO: 13)

MVDPVGFAEAWKAQFPDSEPPRMELRSVGDIEQELERCKASIRRLEQEVN QERFRMIYLQTLLAKEKKSYDRQRWGFRRAAQAPDGASEPRASASRPQPA PADGADPPPAEEPEARPDGEGSPGKARPGTARRPGAAASGERDDRGPPAS VAALRSNFERIRKGHGQPGADAEKPFYVNVEFHHERGLVKVNDKEVSDRI SSLGSQAMQMERKKSQHGAGSSVGDASRPPYRGRSSESSCGVDGDYEDAE LNPRFLKDNLIDANGGSRPPWPPLEYQPYQSIYVGGMMEGEGKGPLLRSQ STSEQEKRLTWPRRSYSPRSFEDCGGGYTPDCSSNENLTSSEEDFSSGQS SRVSPSPTTYRMFRDKSRSPSQNSQQSFDSSSPPTPQCHKRHRHCPWVSE ATIVGVRKTGQIWPNDGEGAFHGDADGSFGTPPGYGCAADRAEEQRRHQD GLPYIDDSPSSSPHLSSKGRGSRDALVSGALESTKASELDLEKGLEMRKW VLSGILASEETYLSHLEALLLPMKPLKAAATTSQPVLTSQQIETIFFKVP ELYEIHKEFYDGLFPRVQQWSHQQRVGDLFQKLASQLGVYRAFVDNYGVA MEMAEKCCQANAQFAEISENLRARSNKDAKDPTTKNSLETLLYKPVDRVT RSTLVLHDLLKHTPASHPDHPLLQDALRISQNFLSSINEEITPRRQSMTV KKGEHRQLLKDSFMVELVEGARKLRHVFLFTDLLLCTKLKKQSGGKTQQY DCKWYIPLTDLSFQMVDELEAVPNIPLVPDEELDALKIKISQIKSDIQRE KRANKGSKATERLKKKLSEQESLLLLMSPSMAFRVHSRNGKSYTFLISSD YERAEWRENIREQQKKCFRSFSLTSVELQMLTNSCVKLQTVHSIPLTINK EDDESPGLYGFLNVIVHSATGFKQSSKALQRPVASDFEPQGLSEAARWNS KENLLAGPSENDPNLFVALYDFVASGDNTLSITKGEKLRVLGYNHNGEWC EAQTKNGQGWVPSNYITPVNSLEKHSWYHGPVSRNAAEYLLSSGINGSFL VRESESSPGQRSISLRYEGRVYHYRINTASDGKLYVSSESRFNTLAELVH HHSTVADGLITTLHYPAPKRNKPTVYGVSPNYDKWEMERTDITMKHKLGG GQYGEVYEGVWKKYSLTVAVKTLKEDTMEVEEFLKEAAVMKEIKHPNLVQ LLGVCTREPPFYIITEFMTYGNLLDYLRECNRQEVNAWLLYMATQISSAM EYLEKKNFIHRDLAARNCLVGENHLVKVADFGLSRLMTGDTYTAHAGAKF PIKWTAPESLAYNKFSIKSDVWAFGVLLWEIATYGMSPYPGIDLSQVYEL LEKDYRMERPEGCPEKVYELMRACWQWNPSDRPSFAEIHQAFETMFQESS ISDEVEKELGKQGVRGAVSTLLQAPELPTKTRTSRRAAEHRDTTDVPEMP HSKGQGESDPLDHEPAVSPLLPRKERGPPEGGLNEDERLLPKDKKTNLFS ALIKKKKKTAPTPPKRSSSFREMDGQPERRGAGEEEGRDISNGALAFTPL DTADPAKSPKPSNGAGVPNGALRESGGSGFRSPHLWKKSSTLTSSRLATG EEEGGGSSSKRFLRSCSASCVPHGAKDTEWRSVTLPRDLQSTGRQFDSST FGGHKSEKPALPRKRAGENRSDQVTRGTVTPPPRLVKKNEEAADEVFKDI MESSPGSSPPNLTPKPLRRQVTVAPASGLPHKEEAGKGSALGTPAAAEPV TPTSKAGSGAPGGTSKGPAEESRVRRHKHSSESPGRDKGKLSRLKPAPPP PPAASAGKAGGKPSQSPSQEAAGEAVLGAKTKATSLVDAVNSDAAKPSQP GEGLKKPVLPATPKPQSAKPSGTPISPAPVPSTLPSASSALAGDQPSSTA FIPLISTRVSLRKTRQPPERIASGAITKGWLDSTEALCLAISRNSEQMAS HSAVLEAGKNLYSFCVSYVDSIQQMRNKFAFREAINKLENNLRELQICPA TAGSGPAATQDFSKLLSSVKEISDIVQREQKLISEEDLEQKLISEEDLEQ KLISEEDLSGSETPGTSESATPESMVSKGEELFTGWPILVELDGDVNGHK FSVSGEGEGDATYGKLTLKFICTTGKLPVPWPTLVTTLTYGVQCFSRYPD HMKQHDFFKSAMPEGYVQERTIFFKDDGNYKTRAEVKFEGDTLVNRIELK GIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTA AGITLGMDELYK

The domains in the sequence above are in order:
p210 BCR-ABL
3xMYC
XTEN L linker
eGFP

### Example 2 - EXOAMP in Vivo Targeting of CD19 Expressing NALM6 Cells and Intracellular Expressing GFP NALM6 Cells in a Subcutaneous NOD Scid Gamma (NSG) Mouse by αCD19-CAR and EXOAMP GFP-CAR Expressing Bispecific Protein Targeting GFP/CD19 and Non-Transduced (NT) T-Cells

In this example the results show that the EXOAMP GFP CAR targets and lyses double positive CD19/eGFP expressing NALM6.3xMYC-XTEN_L-eGFP tumour, while sparing single positive NALM6 tumour in bilaterally flanked NSG mice.

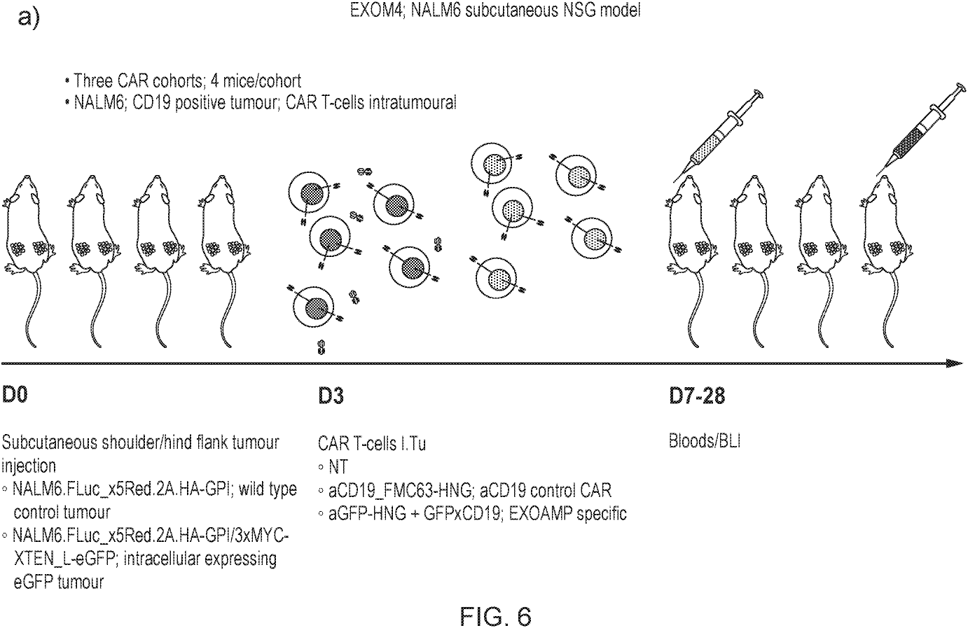

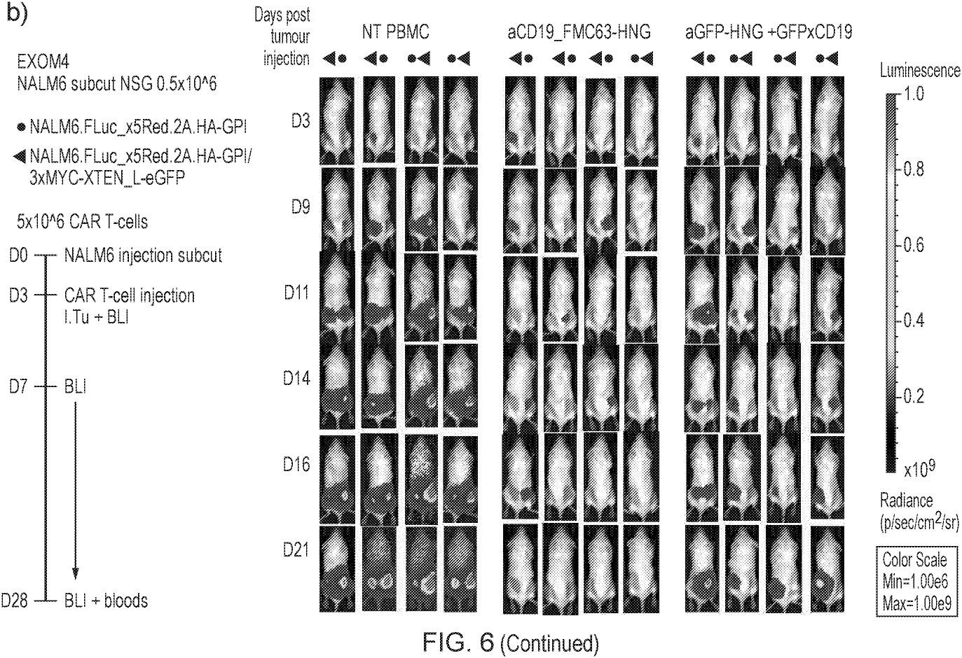

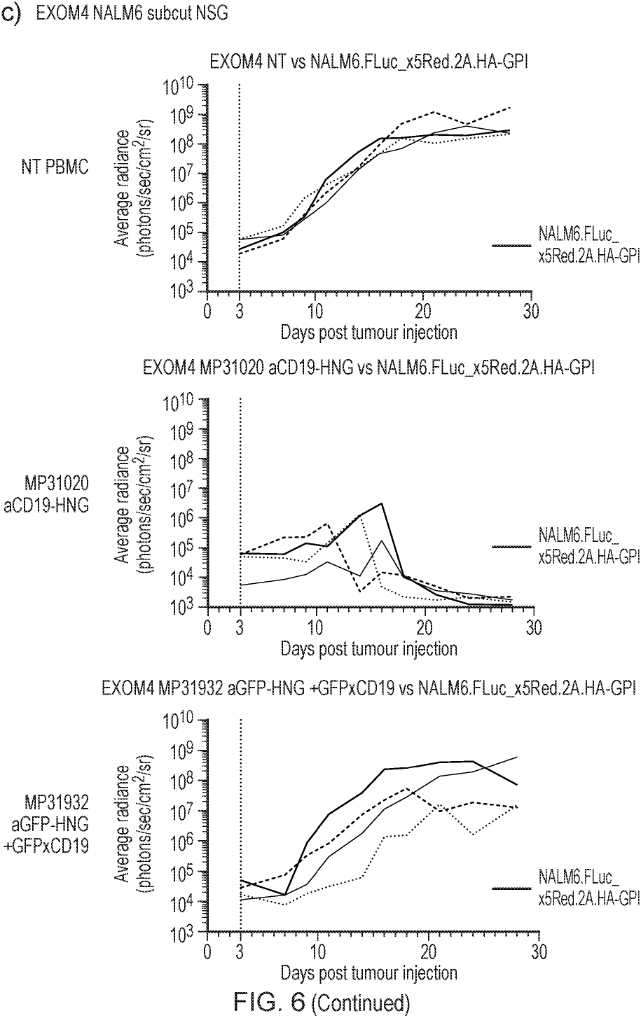

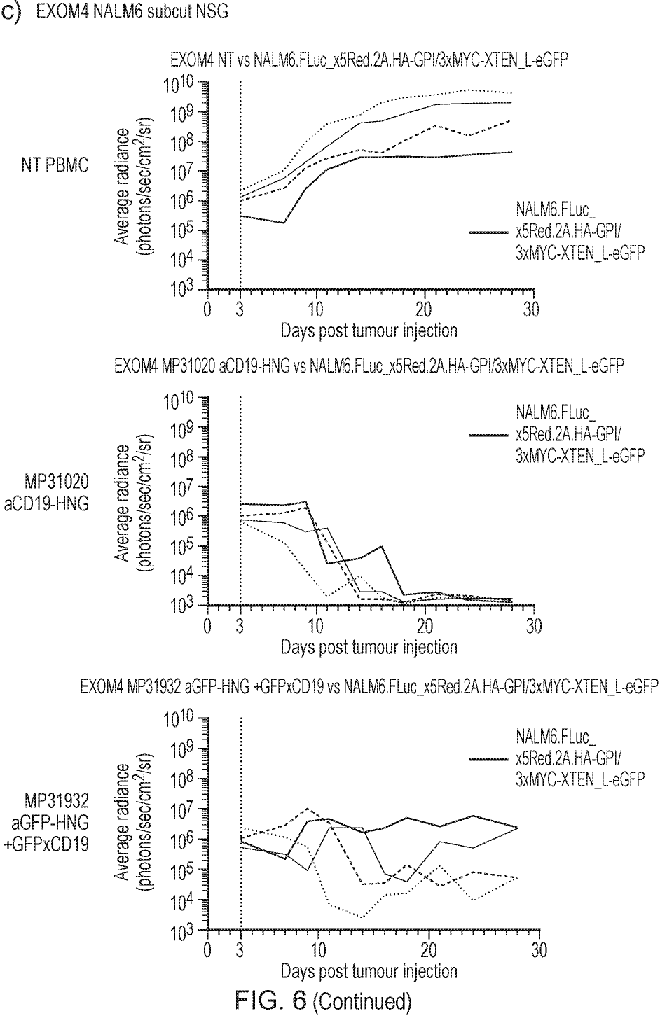

FIG. 6 a) shows details of timeline, cohorts, sampling and routes of injection in the in vivo NALM6 subcutaneous animal model. NALM6 is a B cell precursor leukaemia cell line. It is a model of acute lymphoblastic leukaemia; is CD19-positive and CD33-negative.

NSG mice were injected with a control tumour (0.5x10^6 NALM6 [NALM6.Fluc_x5Red.2A.HA-GPI]) and an intracellular expressing eGFP tumour (NALM6.3xMYC-XTEN_L-eGFP [NALM6.FLuc_x5Red.2A.HA-GPI/3xMYC-XTEN_L-eGFP] on alternating hind flanks [i.e. different tumour cells were injected into each flank resulting in the development of different tumours on each flank of the mouse as shown in FIG. 6b)].

The tumour cells had been transduced to firefly luciferase/ glycosylphosphatidylinositol anchored HA tag (Fluc_xRed.2A.HA-GPI) for use as a marker for tumour growth in vitro and in vivo via bioluminescent imaging.

Three days after injection of the tumour cells, 5x10^6 CAR T-cells were injected intratumourally.

The CAR T- cells were either aCD19_FMC63-HNG - αCD19 control CAR; or αGFP-HNG + GFPxCD19; EXOAMP specific CAR.

Raw images from D3-21 are shown in FIG. 6b) and show tumour control of both tumours using the αCD19-CAR; and specific double positive NALM6.3xMYC-XTEN_L-eGFP tumour control using the EXOAMP GFP CAR.

For example, in FIG. 6b) the NT PBMC panel of photographs on the left hand side shows continual tumour development from day 3 to 21 on both flanks. The aCD19-CAR_FMC63-HNG panel of photographs in the middle of the figure shows control of the two different tumour types since no or small tumours are present on the flanks which have been injected with both types of tumour cell. The EXOAMP (GFP-CAR NALM6.3xMYC-XTEN_L-eGFP) panel of photographs on the right hand side of the figure show that this CAR controlled growth of the double positive NALM6.3xMYC-XTEN_L-eGFP tumour only. It can be seen that the flank which was injected with 0.5x10^6 NALM6 tumour cells continues to grow when treated with the αGFP-HNG+GFPxCD19 CAR.

This data is further plotted with average radiance at the tumour site in FIG. 6c), which demonstrates specific tumour control with the EXOAMP GFP-CAR only.

The solid lines indicate specific tumour average radiance of NALM6 (see graphs on the left hand side of the figure) and NALM6.3xMYC-XTEN_L-eGFP (see graphs on the right hand side of the figure). The lines indicate average readings of each subject mouse/cohort.

An exemplary sequence used in this example is:

intracellular 3xMYC-XTEN_L-eGFP (SEQ ID NO: 12)

EQKLISEEDLEQKLISEEDLEQKLISEEDLSGSETPGTSESATPESMVSK
GEELFTGVVPILVELDGDVNGHKFSVSGEGEGDATYGKLTLKFICTTGKL
PVPWPTLVTTLTYGVQCFSRYPDHMKQHDFFKSAMPEGYVQERTIFFKDD
GNYKTRAEVKFEGDTLVNRIELKGIDFKEDGNILGHKLEYNYNSHNVYIM
ADKQKNGIKVNFKIRHNIEDGSVQLADHYQQNTPIGDGPVLLPDNHYLST
QSALSKDPNEKRDHMVLLEFVTAAGITLGMDELYK

The domains in the sequence above are in order:
3xMYC
XTEN L linker
eGFP

### Example 3 - EXOAMP in Vivo Targeting of CD19 Expressing NALM6 Cells and Intracellular Expressing GFP NALM6 Cells in a Subcutaneous NOD Scid Gamma (NSG) Mouse by αCD19-CAR and EXOAMP GFP-CAR Expressing Either- Bispecific Protein Targeting GFP/CD19 or an Irrelevant Protein Targeting GFP/CD33 and Non-Transduced (NT) T-Cells

In this example the results show that EXOAMP GFP CAR +GFPxCD19 targets and lyses double positive CD19/eGFP expressing NALM6.3xMYC-XTEN_L-eGFP tumour, while sparing single positive NALM6 tumour in bilaterally flanked NSG mice and the GFP-CAR +GFPxCD33 fails to control both tumours similarly to NT T-cells.

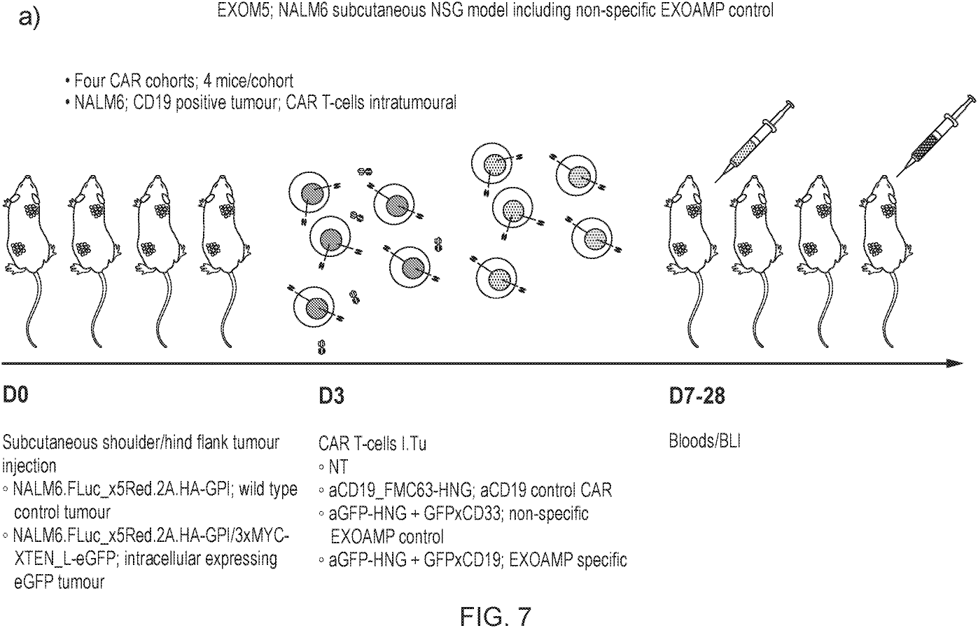

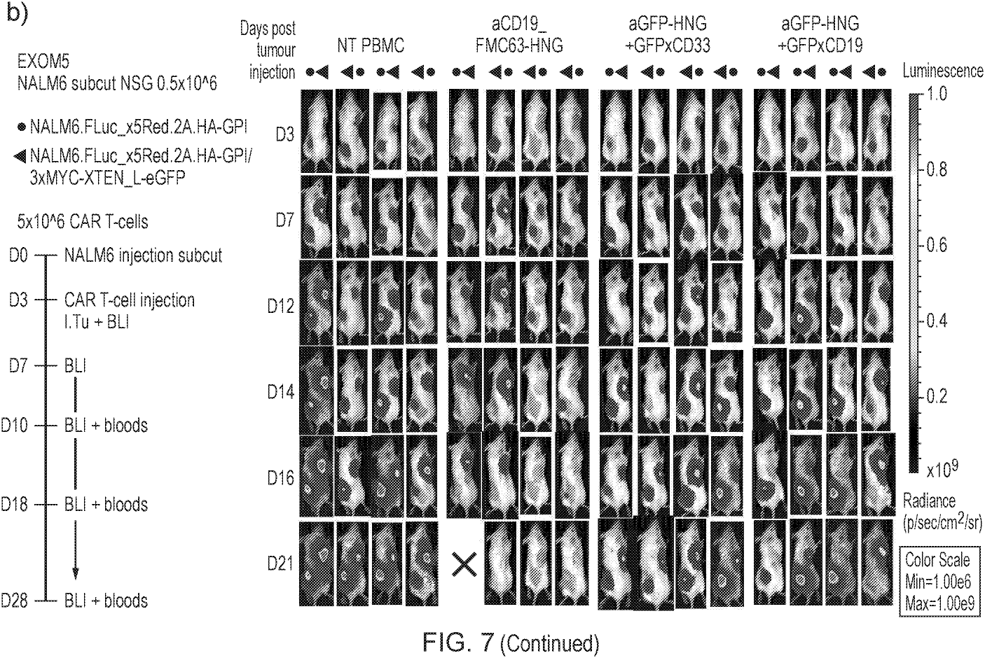

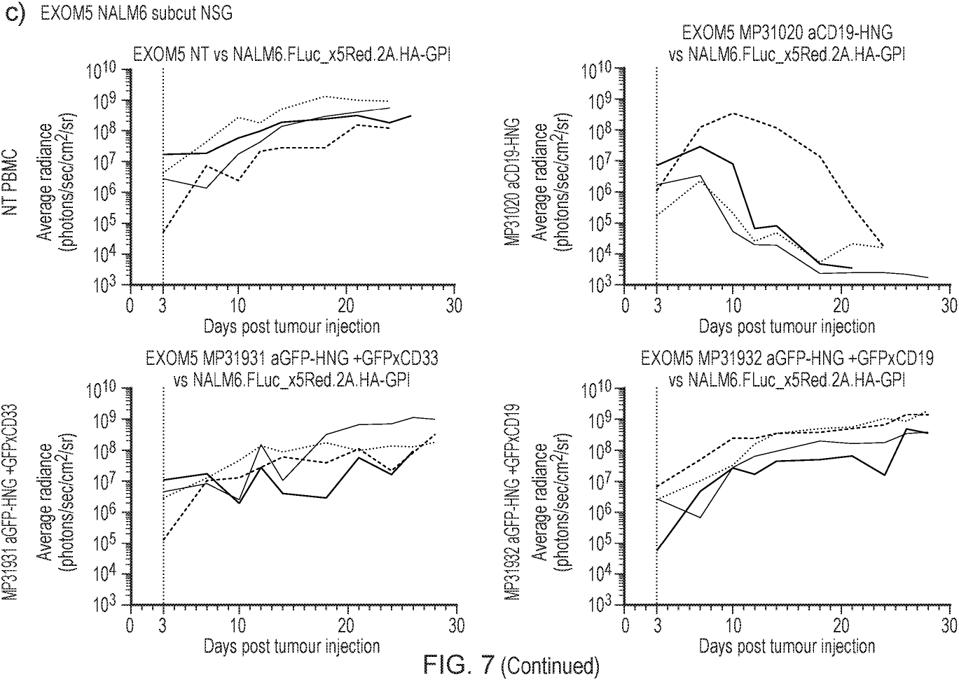

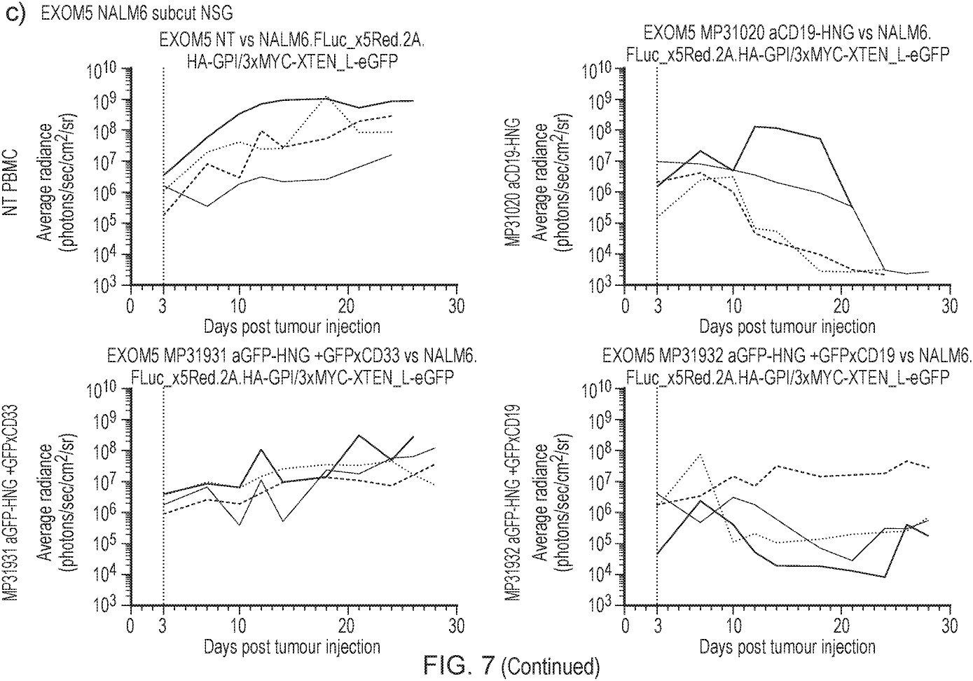

FIG. 7 a) shows details of timeline, cohorts, sampling and routes of injection in the in vivo NALM6 subcutaneous animal model.

NSG mice were injected with a control tumour (0.5x10^6 NALM6 [NALM6.Fluc_x5Red.2A.HA-GPI]) and an intracellular expressing eGFP tumour (NALM6.3xMYC-XTEN_L-eGFP [NALM6.FLuc_x5Red.2A.HA-GPI/3xMYC-XTEN_L-eGFP] on tumour on alternating sites - left hind flank and right shoulder [i.e. different tumour cells were injected into each site resulting in the development of different tumours at each site as shown in FIG. 7 b)].

The tumour cells had been transduced to express firefly luciferase/ glycosylphosphatidylinositol anchored HA tag (Fluc_xRed.2A.HA-GPI) for use as a marker for tumour growth in vitro and in vivo via bioluminescent imaging.

Raw images from D3-21 are shown in FIG. 7b and show control of both tumours by the αCD19-CAR (see the second panel of photographs in the figure which shows that tumours at both injection sites are controlled over time); and specific double positive NALM6.3xMYC-XTEN_L-eGFP tumour control in the EXOAMP GFP CAR (see the fourth panel of photographs in the figure which show that only the NALM6.3xMYC-XTEN_L-eGFP tumour is controlled. The NALM6.FLuc_x5Red.2A.HA-GPI tumour continues to grow when treated with αGFP-HNG+GFPxCD19). The GFP-CAR _GFPxCD33 fails to control both tumours (see the third panel of photographs in the figure which shows that tumours grow at both injection sites. The CAR cannot target the tumour because it is CD33-negative). The NT T-cells are unable to control both tumours (see the first panel of photographs in the figure which shows that tumours grow at both injection sites).

This data is further plotted with average radiance at the tumour site in FIG. 7 c), demonstrating specific tumour control with the EXOAMP GFP-CAR +GFPxCD19 only.

The solid lines indicate specific tumour average radiance of NALM6 (see the left hand side of the figure) and NALM6.3xMYC-XTEN_L-eGFP (see the right hand side of the figure). The lines indicate average readings of each subject mouse/cohort. The first mouse of cohort 2 (αCD19_FMC63- HNG) had to be culled due to weight loss before D21.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

### Example 4 - Sandwich ELISA for Detection of Extracellular P53 In Vitro From Cellular Supernatant

To detect extracellular p53 in vitro in cellular supernatant, colorectal cancer cell lines- RKO, HT-29, LS123, LS174T, SW1463 and CL-40- were harvested from confluent flasks and washed twice in media and plated in a flat bottomed 96-well plate at 1×10⁵ cells/well in 200 µl/well. The plates were maintained at 37° C., /5% CO₂ for 48 hours, after which 100 µl supernatant was harvested and frozen at -20° C. until use.

For the ELISA, 2 µg/ml pan-p53 antibody DO-1 clone was coated at 100 µl/well in 0.2 M sodium carbonate/bicarbonate buffer pH 9.6 in a Nunc MaxiSorp™ flat-bottom overnight. The next day, plates were washed and blocked with ELISA Assay Diluent (ADA; BioLegend, Inc) for 1 hour with gentle shaking. The plates were then washed and incubated with 100 µl cell supernatant for 2 hour with gently shaking. The plates were washed and polyclonal rabbit anti-p53 antibody (Life Technologies) was incubated onto the wells at 1:2000 dilution at 100 µl/well in ADA for 1 hours with gentle shaking. Plates were washed and incubated secondary antibody horseradish peroxidase-conjugated donkey anti-rabbit IgG (Biolegend, Inc) at a 1:2000 dilution in ADA for 30 minutes with gentle shaking. Plates were washed in PBS 0.05% Tween-20 five times with 30 second soak. Plates were incubated with 100 µl/well TMB substrate (BioLegend, Inc) in the dark, and stopped using 1 M sulphuric acid solution. Absorbance was measured at 450 and 570 nM.

Control cells, PBMCs were either, activated using 0.5 ug/ml CD3/CD28 antibodies and 100 IU/ml IL-2, or non-activated, and plated at 1×10⁵ cells/well in 200 µl/well in a flat-bottomed 96 well plate.

Purified p53 protein was used as a standard, from 8000 pg/ml and diluted 1:2 sequentially.

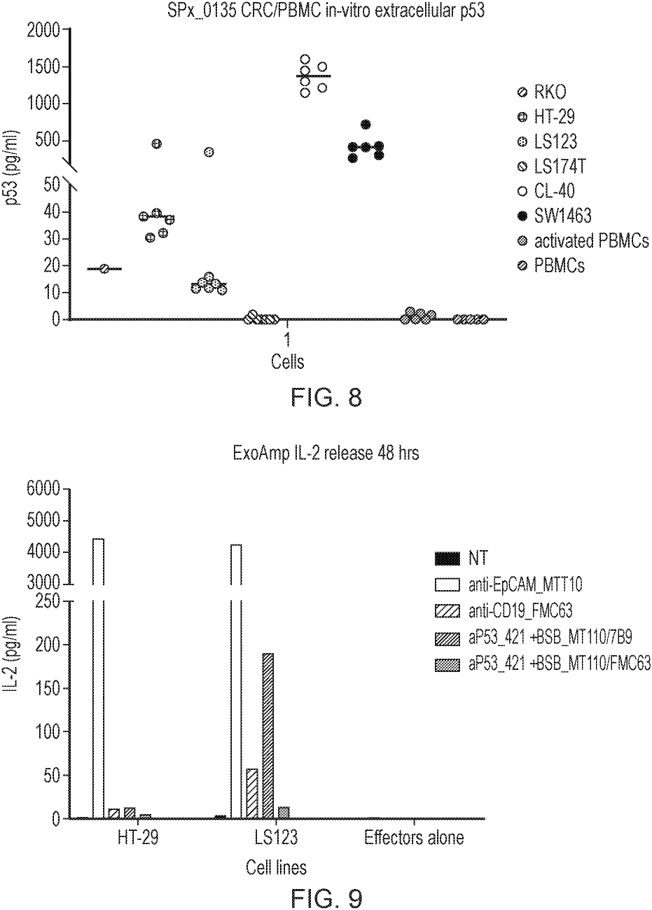

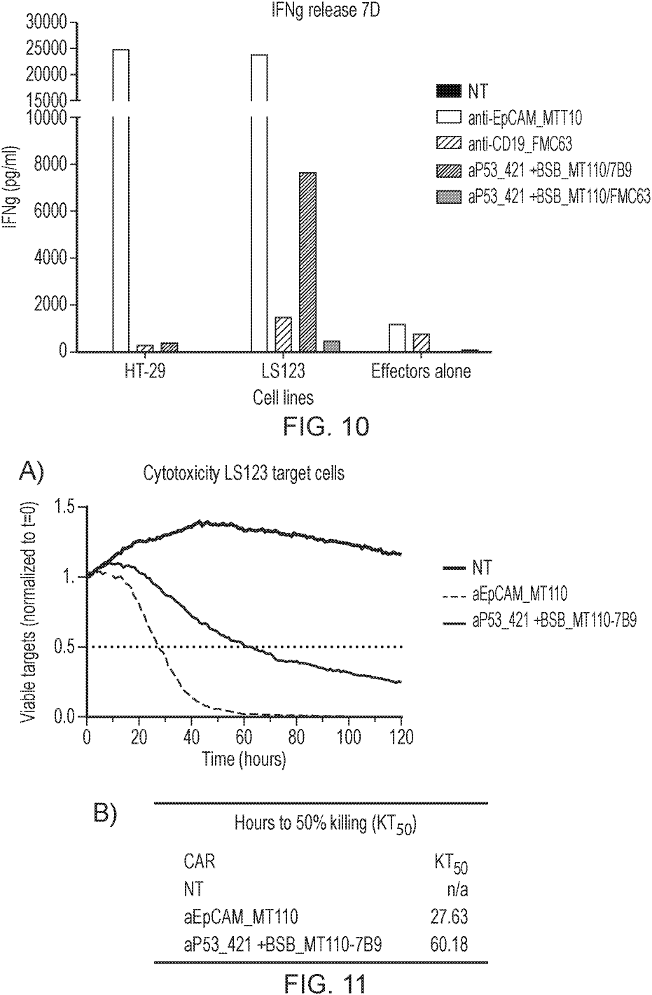

FIG. 8 shows that extracellular p53 protein can be detected in the supernatant of 5 our of 6 of colon cancer cell lines tested

### Example 5 - EXOAMP Specific Targeting of Colorectal Cancer Cells Expressing Cell Surface EpCAM and p53 Mutant R175H

IL-2 and IFNy cytokine release was measured from CAR T cells or non transduced cells after culture with target colorectal cancer cells HT-29 (which express cell surface EpCAM and a R273H mutation in p53) orS123 (which express cell surface EpCAM and a R175H mutation in p53).

The sequences of the variable domains of each antibody clone were obtained from publicly available sequences and ordered as human codon optimized gblocks (Integrated DNA Technologies, Inc) as a single chain variable fragment (scFv) containing Ig kappa chain signal peptide in either VL/VH or VL/VH orientation separated by a flexible linker (Gly₄Ser)₄ or (Gly₄Ser)₃. The gblock was PCR amplified using Phusion® High-Fidelity DNA Polymerase (New England Biolabs) as per the manufacturer’s protocol and sub cloned into a modified SFG retroviral vector-containing a scaffold attachment region- in frame with human IgG1 hinge, CD28 transmembrane and intracellular 41BB and CD3Z domains and downstream from sort-suicide gene RQR8 separated by a Thosea asigna 2A peptide sequence.

The variable domain sequences of the anti-EpCAM binder MT110 and the various anti-p53/anti-CD19 binders were obtained from publically available sequences and ordered as human codon optimized gblocks (Integrated DNA Technologies, Inc) with an N-terminal IL-2 signal peptide and intervening flexible 15aa linker (Gly₄Ser)₃. A C-terminal serine-glycine linker Gly₄Ser and hexahistidine tag was added using modified primers and amplified as PCR fragments and sub cloned into a modified SFG vector downstream of the CAR, separated by an equine 2A peptide.

To measure target specific IL-2 and IFNγ release from CAR effectors and target cells colorectal cancer target cells were plated into a 96-well flat bottomed plate at a concentration of 1×10⁵ cells per 200 µl for 18-24 hours, or until attached. Subsequently, 100 µl of media was removed and 5×10⁴ CAR T-cells or control cells in 100 µl were added to each well and incubated for 2 days for IL-2 measurement or 7 days for IFNγ measurement. 100 µl of media was removed and cytokine concentration was assay using IL-2 or IFNγ specific ELISA.

### Results

FIG. 9 shows IL-2 cytokine release from pantropic p53 CAR (aP53_421) expressing T cells secreting a bispecific binder against EpCAM and mutant p53 R175H (+BSB_MT110/7B9) against LS123 cells (which express cell surface EpCAM and harbour an R175H mutation in p53).

T cells comprising an anti-CD19_FMC63 CAR or an anti-p53 CAR in combination with a bispecific binder targeting EpCAM/CD19 (aP53_421+BSB_MT110/FMC63) were used as negative controls. These cells generated low or background levels of IL-2 comparable to non-transduced cells against HT-29 and LS123 targets.

T cells comprising an anti-EpCAM targeting CAR (anti-EpCAM_MT110) were used as a positive control. These cells secreted 4428 pg/ml and 4249 pg/ml IL-2 against colorectal target cells HT-29 and LS123 respectively.

T cells comprising an ExoAmp CAR targeting p53 and secreting a bispecific binder against EpCAM and mutant p53 R175H (aP53_421 +BSB_MT110/7B9) secreted IL-2 against R175H mutant p53 cell line LS123 at about 189 pg/ml. FIG. 9 shows that these T cells secreted very low or background levels of IL-2 against cell line HT-29 which does not express the p53 mutant R175H.

Levels of IL-2 were undetectable in the absence of targets (effectors alone) for all CARs tested.

FIG. 10 shows IFNγ cytokine release from pantropic p53 CAR (aP53_421) expressing T cells secreting a bispecific binder against EpCAM and mutant p53 R175H (+BSB_MT110/7B9) against LS123 cells (which express cell surface EpCAM, and harbour a R175H mutation in p53.

T cells comprising an anti-CD19_FMC63 CAR or an anti-p53 CAR in combination with a bispecific binder targeting EpCAM/CD19 (aP53 421+BSB_MT110/FMC63) were used as negative controls. These cells generated low or background levels of IFNγ, comparable to non-transduced cells against HT-29 and LS123 targets.

T cells comprising an anti-EpCAM targeting CAR (anti-EpCAM_MT110) were used as a positive control. These cells secreted 23735 pg/ml and 22553 pg/ml IFNγ against colorectal target cells HT-29 and LS123 respectively.

T cells comprising an ExoAmp CAR targeting p53 and secreting a bispecific binder against EpCAM and mutant p53 R175H (aP53_421 +BSB_MT110/7B9) secreted IFNγ against R175H mutant p53 cell line LS123 at about 7625 pg/ml. FIG. 10 shows that these T cells secreted very low or background levels of IFNγ against cell line HT-29 which does not express the p53 mutant R175H.

Levels of IFN-γ were undetectable or at very low/background level in the absence of targets (effectors alone) for all CARs tested.

Exemplary sequences used in this example are:

Anti-EpCAM_MT110 - Anti-EpCAM CAR co-expressing RQR8 marker gene, with anti-EpCAM MT110 scFv_LH orientation, IgG1 hinge spacer, CD28TM domain and 41BB and CD3Z intracellular domains (SEQ ID NO: 14).

Anti-CD19_FMC63 - anti-CD19 CAR co-expressing RQR8 marker gene, with anti-CD19 FMC63 scFv LH orientation, IgG1 hinge spacer, CD28TM domain and 41BB and CD3Z intracellular domains. (SEQ ID NO: 2)

aP53_421 +BSB_MT110/7B9 - Anti-p53 CAR co-expressing RQR8 marker gene, with anti-p53 pAb421 scFv LH orientation, IgG1 hinge spacer, CD28TM domain and 41BB and CD3Z intracellular domains; and hexahistidine tagged bispecific binder targeting EpCAM (MT110_LH) and mutp53 R175H (7B9_HL) (SEQ ID NO: 15).

aP53_421 +BSB_MT110/FMC63 - Anti-p53 CAR co-expressing RQR8 marker gene, with anti-p53 pAb421 scFv LH orientation, IgG1 hinge spacer, CD28TM domain and 41BB and CD3Z intracellular domains; and hexahistidine tagged bispecific binder targeting EpCAM (MT110_LH) and CD19 (FMC63_HL) (SEQ ID NO: 16).

### Example 6 - EXOAMP In Vitro Cytotoxicity Assay

An in vitro real time cytotoxicity killing assay was performed. mKate2 positive colorectal cancer target cells- LS123- were plated in 96-well flat bottomed plates at a concentration of 2×10⁴ cells per 200 µl with 50:50 media/conditioned media (from growing LS123 cells) for 18-24 hours, or until attached. Subsequently, 50 µl of media was removed and 8×10⁴ CAR T-cells or control cells in 50 µl were added to each well at a 4:1 E:T (CAR:target) ratio, and incubated for 120 hours and assayed on the IncuCyte S3 Live-Cell Analysis System (Essen Biotech). Two images were taken per well, every hour at 10x magnification. Data was analysed using the IncuCyte® Zoom software to detect and count number of mKate2 positive nuclei per image.

### Results

FIG. 11 shows in vitro real time cytotoxicity of pantropic p53 CAR (aP53_421) expressing T-cells secreting a bispecific binder against EpCAM and mutant p53 R175H (+BSB_MT110/7B9) against LS123 cells which express cell surface EpCAM, and harbour mutant p53 R175H. Control effectors include non-transduced cells (NT) and positive control CAR anti-EpCAM (aEpCAM_MT110).

FIG. 11 shows that the ExoAmp specific CAR aP53_421 +BSB_MT110-7B9 was able to lyse target LS123 cells specifically. The amount of time taken to kill 50% of the target cells (KT50) of the ExoAmp specific CAR aP53_421 +BSB_MT110-7B9 was 60.18 hours, compared to positive control aEpCAM_MT110, which had a KT50 of 27.63 hours.

1. A cell which comprises;
(i) a chimeric antigen receptor (CAR) comprising a binding domain which binds a first epitope of a tumour antigen; and
(ii) a polynucleotide which encodes a bi-specific protein which comprises:
a first binding domain which binds a second epitope of said tumour antigen; and
a second binding domain which binds a cell surface antigen.

2. A cell according to claim 1, wherein the cell surface antigen is a cell surface tissue antigen.

3. A cell according to any preceding claim, wherein the tumour antigen is not a cell surface tumour antigen, preferably said tumour antigen does not comprise a transmembrane domain; a lipid anchor such as a glycosylphosphatidylinositol (GPI)-anchor.

4. A cell according to any preceding claim, wherein the tumour antigen does not comprise a signal peptide.

5. A cell according to any preceding claim, wherein the tumour antigen is expressed at a higher level in the tumour compared with a corresponding, non-cancerous tissue, or wherein the antigen is tumour-specific.

6. A cell according to any preceding claim, wherein the first or second epitope of the tumour antigen comprises a tumour-specific mutation.

7. A cell according to claim 6, wherein the mutation is selected from a substitution, insertion or deletion.

8. A cell according to any preceding claim, wherein the tumour antigen is a fusion protein, suitably said fusion protein may comprise at least two domains, wherein a first domain comprises the first epitope of a tumour antigen and a second domain comprises the second epitope of the tumour antigen.

9. A cell according to any preceding claim, wherein the first or second epitope of the tumour antigen comprises a tumour-specific post-translational modification.

10. A cell according to claim 9, wherein the tumour-specific post-translational modification is phosphorylation, suitably one of the tumour antigen epitopes may be the phosphorylation site.

11. A cell according to any preceding claim, wherein:
a) the binding domain of the CAR binds a first epitope of a tumour antigen which is specific to the tumour; and/or
b) the first binding domain of the bi-specific protein binds a second epitope of a tumour antigen which is specific to the tumour.

12. A cell according to any preceding claim wherein the cell is an immune effector cell, such as an alpha-beta T cell, a NK cell, a gamma-delta T cell, a cytokine induced killer cell or a macrophage.

13. A nucleic acid construct which comprises:
(i) a first nucleic acid sequence which encodes a CAR comprising a binding domain which binds a first epitope of a tumour antigen; and
(ii) a second nucleic acid sequence which encodes a bi-specific protein which comprises:
a first binding domain which binds a second epitope of said tumour antigen; and
a second binding domain which binds an epitope of a cell surface tissue antigen.

14. A nucleic acid construct according to claim 13, wherein the first and second nucleic acid sequences are separated by a co-expression site.

15. A kit of nucleic acid sequences comprising:
(i) a first nucleic acid sequence which encodes a CAR comprising a binding domain which binds a first epitope of a tumour antigen; and
(ii) a second nucleic acid sequence which encodes a bi-specific protein, which comprises:
a first binding domain which binds a second epitope of said tumour antigen; and
a second binding domain which binds an epitope of a cell surface tissue antigen.

16. A vector which comprises a nucleic acid construct according to claim 13 or 14.

17. A kit of vectors which comprises:
(i) a first vector which comprises a nucleic acid sequence which encodes a CAR comprising a binding domain which binds a first epitope of a tumour antigen; and
(ii) a second vector which comprises a nucleic acid sequence which encodes a bi-specific protein, which comprises:
a first binding domain which binds a second epitope of said tumour antigen; and
a second binding domain which binds an epitope of cell surface tissue antigen.

18. A pharmaceutical composition which comprises a plurality of cells according to any of claims 1 to 12, a nucleic acid construct according to claim 13 or 14, a first nucleic acid sequence and a second nucleic acid sequence as defined in claim 15; a vector according to claim 16 or a first and a second vector as defined in claim 17.

19. A pharmaceutical composition according to claim 18 for use in treating and/or preventing a disease.

20. The pharmaceutical composition for use according to claim 19, wherein the disease is cancer.

21. A method for making a cell according to any of claims 1 to 12, which comprises the step of introducing: a nucleic acid construct according to claim 13 or 14, a first nucleic acid sequence and a second nucleic acid sequence as defined in claim 15; a vector according to claim 16 or a first and a second vector as defined in claim 17 into the cell.

22. A CAR system comprising;
(i) a receptor component comprising a binding domain which binds a first epitope of a tumour antigen, a transmembrane domain and a signaling domain; and
ii) a bi-specific protein which comprises:
a first binding domain which binds a second epitope of said tumour antigen; and
a second binding domain which binds an epitope of a cell surface tissue antigen.

23. A system according to claim 22, wherein said system comprises:
a) an alpha-beta T cell, a NK cell, a gamma-delta T cell, a cytokine induced killer cell or a macrophage which expresses the receptor component; and/or
b) an alpha-beta T cell, a NK cell, a gamma-delta T cell, a cytokine induced killer cell or a macrophage which expresses the bi-specific protein; and/or
c) the receptor component and the bi-specific protein are expressed by the same cell and/or
d) wherein the bi-specific protein is administered to the system.

24. A bi-specific protein for use in treating cancer in combination with a CAR expressing cell, wherein:
the bi-specific protein comprises:
a first binding domain which binds a second epitope of said tumour antigen; and
a second binding domain which binds an epitope of a cell surface tissue antigen; and
the CAR comprises a binding domain which binds a first epitope of a tumour antigen.

25. A CAR expressing cell for use in treating cancer in combination with a bi-specific protein, wherein:
the CAR comprises a binding domain which binds a first epitope of a tumour antigen; and
the bi-specific protein comprises:
a first binding domain which binds a second epitope of said tumour antigen; and
a second binding domain which binds an epitope of a cell surface tissue antigen.
